(19) United States
(12) Patent Application Publication
(10) Pub. No.: US 2026/0142007 A1
(43) Pub. Date: May 21, 2026

(54) DIGITAL THERAPEUTICS AND COMBINATION THERAPY FOR THE TREATMENT OF TENOSYNOVIAL GIANT CELL TUMOR (TGCT)

(71) Applicant: REMEPY HEALTH LTD, Ramat Hasharon (IL)

(72) Inventors: Amir AMEDI, Modi'in - Maccabim Reut (IL); Michal TSUR-SHALEV, Zichron Yaacov (IL); Or SHOVAL, Ramat Hasharon (IL); Nira Neomi SAPORTA, Givatayim (IL); Gutman Renan, Hoboken, NJ (US); Shai Erlich, Woluwe-Saint-Pierre (BE)

(73) Assignee: REMEPY HEALTH LTD, Ramat Hasharon (IL)

(21) Appl. No.: 19/393,668

(22) Filed: Nov. 19, 2025

Related U.S. Application Data
(60) Provisional application No. 63/722,144, filed on Nov. 19, 2024.

(51) Int. Cl. G16H 20/10; G16H 10/60; G16H 15/00; G16H 20/30; G16H 20/60; G16H 20/70

(52) U.S. Cl. CPC G16H 20/10; G16H 10/60; G16H 15/00; G16H 20/30; G16H 20/60; G16H 20/70

(57) ABSTRACT

Provided are methods of digital therapeutics for individuals with benign connective tissue tumor. More specifically, the digital therapeutics are designed to modulate Tenosynovial Giant Cell Tumor (TGCT), on a standalone basis or by combining them with pharmaceutical drugs such as CSF1R inhibitors.

### FIELD OF THE INVENTION

The presently disclosed subject matter relates to the field of digital therapeutics for an individual with benign connective tissue tumor. More specifically, the digital therapeutics are designed to modulate Tenosynovial Giant Cell Tumor (TGCT), on a standalone basis or by combining them with pharmaceutical drugs such as CSF1R inhibitors.

### BACKGROUND

Benign connective tissue tumors encompass a diverse group of non-malignant mesenchymal proliferative disorders arising from fibrous, synovial, adipose, vascular, or tendon-associated tissues, and are frequently characterized by localized cellular overgrowth, inflammatory cell infiltration, and cytokine-driven tissue remodeling that can result in pain, swelling, functional impairment, and progressive structural damage. Among these conditions, Tenosynovial Giant Cell Tumor (TGCT) represents a clinically significant entity originating from the synovium of joints, bursae, and tendon sheaths, occurring in localized and diffuse subtypes that affect small and large joints, respectively. TGCT exhibits synovial-like mononuclear cell proliferation with multinucleated giant cells, foam cells, hemosiderin deposition, and dense infiltrates of macrophages, monocytes, and neutrophils. A recurrent chromosomal translocation, t(1;2)(p13;q37), drives overexpression of colony-stimulating factor 1 (CSF1), resulting in substantial recruitment of macrophages and maintenance of a highly inflammatory microenvironment enriched with cytokines and mediators including interleukin-1β (IL-1β), tumor necrosis factor-α (TNF-α), and receptor activator of nuclear factor-κB ligand (RANKL). These factors promote synovial hypertrophy, angiogenesis, osteoclast differentiation, and joint erosion, contributing to a spectrum of symptoms that include persistent pain, joint swelling, stiffness, reduced range of motion, mechanical catching, instability, and sensations of “giving way.” In diffuse TGCT, extensive tissue infiltration and recurrence drive progressive joint destruction, often culminating in significant disability and the need for repeated surgical intervention or joint replacement. The chronic inflammatory and mechanical burden is further associated with increased rates of comorbidities such as depression, hypertension, and hypothyroidism, amplifying the overall impact of these benign yet locally destructive connective tissue tumors.

The present invention provides digital therapeutic modalities for modulating benign connective tissue tumors, including TGCT, and further enables their use in combination with conventional medicinal agents to achieve synergistic therapeutic effects.

### SUMMARY

In one embodiment the invention provides a method for modulating one or more disease-related markers, symptoms, or disease progression in an individual with benign connective tissue tumor originating from, or causing, dysregulated immune response, the method comprising:
delivering a digitized therapeutic plan to the individual, wherein the digitized therapeutic plan comprises a plurality of therapeutic interventions selected from: at least one physical intervention and at least one psychological intervention;
converting the plurality of therapeutic interventions into a plurality of targeted interaction outputs for presentation to the individual; and
adaptively delivering the digitized therapeutic plan, tracking the individual's responses and associated performance metrics, and continuing until a predetermined condition is met.

In one embodiment the benign connective tissue tumor is Tenosynovial Giant Cell Tumor (TGCT).

In one embodiment the method further comprises obtaining a plurality of attributes associated with the individual from a plurality of data acquisition sources to generate an initial profile for the individual, wherein the digitized therapeutic plan further comprises adaptation based on the initial profile.

In one embodiment the digitized therapeutic plan comprises performing the at least one physical intervention concurrently with the at least one psychological intervention. In one embodiment the digitized therapeutic plan comprises performing the at least one physical intervention concurrently with the at least one psychological intervention, or sequentially, or a combination thereof.

In one embodiment the adaptation further comprises selecting and implementing therapeutic intervention parameters based on at least one attribute, of the plurality of attributes, of the individual selected from: age, hobbies, music preferences, spoken language, caregiver involvement preferences, disease characteristics, tumor location, tumor size, disease severity, the disease-related markers, the symptoms, pain levels, range of motion limitations, joint stiffness, educational background, professional background, and individual-defined goals.

In one embodiment the digitized therapeutic plan is further dynamically adapted based on changes to at least one of the plurality of attributes of the individual during the course of the digitized therapeutic plan.

In one embodiment the method further comprises:
dynamically monitoring the individual's monitored parameters during delivery of the digitized therapeutic plan;
generating a dynamic user profile based on the initial profile and monitored parameters of the individual selected from: Electronic Medical Records (EMR) data, motor attributes, psychological attributes, range of motion measurements, pain levels, joint function, pharmacological attributes, tumor response parameters, disease progression, feedback of the individual, feedback of a medical practitioner, drug intake parameters, or a combination thereof; and
adapting the digitized therapeutic plan based on the dynamic user profile.

In one embodiment the method further comprises recursively adapting the digitized therapeutic plan based on updates to the dynamic user profile, the performance metrics, and the individual's responses.

In one embodiment the recursive adapting of the digitized therapeutic plan comprises dynamically identifying and modifying the therapeutic intervention parameters to implement optimized therapeutic outcomes towards a predetermined therapeutic goal.

In one embodiment the therapeutic intervention parameters comprise any of the following selected from: type, timing, dosage, intensity, sequence, duration, frequency, mode of delivery, level of guidance, feedback modality, language adaptation, complexity level, interface configuration, integration with external systems, or a combination thereof, of one or more of the therapeutic interventions.

In one embodiment the recursive adapting is performed using a feedback control algorithm, Bayesian inference, or a combination thereof.

In one embodiment the at least one physical intervention is selected from: physiotherapy, range-of-motion exercises, mobility training, limb agility exercises, functional movement training, balance exercises, proprioception exercises, postural stability training, strength training, stretching exercises, coordination training, low impact training, aerobics exercises, flexibility training, and walking, or a combination thereof.

In one embodiment the at least one psychological intervention is selected from: guided imagery, psychoeducation, psychotherapy, cognitive behavioral therapy, stress and anxiety management training, mindfulness-based interventions, body scanning training, sleep hygiene, fatigue training, pain psychology, pacing training, acceptance and commitment therapy (ACT), dialectical behavior therapy (DBT), attention training techniques, psychodynamic therapy, solution-focused brief therapy (SFBT), narrative therapy, pain therapy, addiction therapy, gestalt therapy, behavioral activation therapy, adjustment therapy, grief therapy, motivational therapy, meaning-centered therapy, creative therapy, expressive therapy, logotherapy, telepsychiatry, and teletherapy, or a combination thereof.

In one embodiment each of the plurality of therapeutic interventions has a duration ranging between 10 s to 60 mins.

In one embodiment the predetermined condition is selected from: a predetermined number of iterations, a predetermined duration has elapsed, a predetermined number of therapeutic interventions have been completed, predetermined progress measurement, predetermined regression measurement, target threshold score achieved, transition to maintenance therapy, and no further improvement is measured, or a combination thereof.

In one embodiment the targeted interaction outputs are selected from: visual, auditory, tactile, or a combination thereof.

In one embodiment the plurality of therapeutic interventions further comprise: sensory inhibition, sensory substitution, sensory integration, or a combination thereof.

In one embodiment the method further comprises pairing a conditioned stimulus with at least one therapeutic intervention such that subsequent presentation of the conditioned stimulus elicits a therapeutic response associated with the intervention.

In one embodiment the method further comprises implementing a nutrition regimen in the individual.

In one embodiment the nutrition regimen comprises any of the following selected from: portion sizing to maintain healthy body weight, meal timing, anti-inflammatory diet, joint-supportive nutrients, weight management support, reduction of pro-inflammatory food intake, hydration, Mediterranean diet, or a combination thereof.

In one embodiment the disease-related markers comprise any of the following selected from: tumor size, tumor spread, objective response rate (ORR), inflammation, macrophage recruitment or activity, cytokine levels, angiogenesis, tumor cell proliferation, fibrosis, joint swelling, pain, stiffness, physical function, range of motion, quality of life, patient perception of disease, psychological endpoints, grip strength, and gait stability.

In one embodiment the benign connective tissue tumor is selected from: TGCT, lipoma, angiolipoma, fibrolipoma, spindle cell lipoma, pleomorphic lipoma, hibernoma, fibroma, desmoid tumor, nodular fasciitis, palmar fibromatosis, plantar fibromatosis, elastofibroma dorsi, myofibroma, leiomyoma, angioleiomyoma, rhabdomyoma, schwannoma, neurofibroma, perineurioma, traumatic neuroma, hemangioma, lymphangioma, glomus tumor, myxoma, intramuscular myxoma, granular cell tumor, solitary fibrous tumor, Giant Cell Tumor of Bone (GCTB), histiocytic disorders, lipoma arborescens, and synovial chondromatosis.

In one embodiment the method further comprises administering at least one medicinal agent before, during, after, or a combination thereof, the implementation of at least one of the therapeutic interventions.

In one embodiment the at least one medicinal agent comprises any of the following selected from: CSF1R-inhibitor, anti-inflammatory drug, immunomodulatory drug, cytokine-targeting biologic, nirogacestat, denosumab, everolimus, methotrexate, and corticosteroids or a combination thereof.

In one embodiment the CSF1R-inhibitors comprises any of the following selected from: Pimicotinib, Pexidartinib, Vimseltinib,, BLZ945 (Sotuletinib), JNJ-40346527 (Edicotinib), IACS-9439, BPR1R024, ABT-869 (linifanib), Imatinib, AG013736 (axitinib), SU11248 (sunitinib), BAY 43-9006 (sorafenib), CHIR258 (tandutinib), Ki20227, Emactuzumab (RG7155), Lacnotuzumab (MCS110), Cabiralizumab (FPA008), PD-036032, ARRY-382 (PF-07265804), EI-1071, TE-952 (JTE-952), PLX5622, GW2580, IMC-CS4 (LY3022855), and AMG820 or a combination thereof.

In one embodiment the method further comprises the administration of an adjunct agent comprising any of the following selected from: nonsteroidal anti-inflammatory drugs (NSAIDS), immunomodulatory agent, cytokine-targeting biologic, corticosteroids, analgesics, anesthetic agents, or a combination thereof.

In one embodiment the method further comprises a conditioning stimulus delivered before, during, after, or a combination thereof, the administering of the at least one medicinal agent, at least one of the therapeutic interventions, or a combination thereof.

In one embodiment the method further comprises:
obtaining a plurality of attributes associated with the individual from a plurality of data acquisition sources to generate an initial profile for the individual, wherein the digitized therapeutic plan further comprises adaptation based on the initial profile;
wherein the adaptation further comprises selecting and implementing therapeutic intervention parameters based on at least one attribute of the individual selected from: age, hobbies, music preferences, spoken language, caregiver involvement preferences, disease characteristics, tumor location, tumor size, disease severity, disease-related markers, symptoms, pain levels, range of motion limitations, joint stiffness, educational background, professional background, medicinal agent intake parameters, and individual-defined goals.

In one embodiment the invention provides a digital therapeutic system for modulating one or more disease-related markers, symptoms, or disease progression in an individual with benign connective tissue tumor originating from, or causing, dysregulated immune response, the system comprising:
a data-input module configured to onboard the individual and dynamically monitor and store data related to the individual throughout a digitized therapeutic plan; and
a digital therapy delivery module comprising a processor configured to deliver the digitized therapeutic plan comprising a plurality of therapeutic interventions selected from: at least one physical intervention and at least one psychological intervention;
wherein the digital therapy delivery module is further configured to convert the digitized therapeutic plan into a plurality of targeted interaction outputs for presentation to the individual and track the individual's responses.

In one embodiment the digital therapy delivery module comprises software executable on a personal electronic device.

In one embodiment the data-input module is configured to receive data from a plurality of data acquisition sources in real-time, wherein the data comprises one or more of: Electronic Medical Records (EMR) data, motion sensor data, position sensor data, environmental sensors data, audio sensors data, location sensors data, audio recording data, speech recognition data, gaze-tracking data, touch interaction data, physiological sensor data, optical sensor, imaging sensors, self-report data, or a combination thereof.

In one embodiment the digital therapy delivery module comprises any of the following selected from: a personal electronic device, a graphical user interface, audio interface, haptic interface, or a combination thereof.

In one embodiment the processor is further configured to:
dynamically monitor any of the following factors of the individual selected from: Electronic Medical Records (EMR) data, motor attributes, psychological attributes, cognitive attributes, pharmacological attributes, and drug intake parameters, or a combination thereof, throughout the implementation of the digitized therapeutic plan;
generate a dynamic user profile, and recursively adapt the digitized therapeutic plan based on the dynamic user profile, performance metrics, and the individual's responses;
or a combination thereof.

In one embodiment the system further comprises at least one additional device operatively coupled to the digital therapy delivery module, selected from: health monitoring systems, medical devices, haptic devices, external speakers, headphones, virtual reality headsets, augmented reality glasses or devices, biofeedback sensors, wearable activity trackers, smartphones, tablets, smartwatches, personal computational devices, smart speakers, voice assistants, motion tracking sensors, virtual assistant systems, internet hubs, gait sensors, eye-tracking devices, voice recording devices, and wearable devices, or combinations thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A and 1B show representations of Center for Epidemiologic Studies Depression (CES-D) scores in subjects pre-and post-intervention i.e., decrease in depression scores with the intervention of the invention; for subjects with Subjective Cognitive Decline (SCD).

FIG. 2A shows intra-network connectivity in the brain. FIG. 2B shows a matrix showing significant connectivity differences between Default Mode Network (DMN) and Salience Network (SN) brain regions.

FIG. 2C shows a graphical representation of changes in DMN-SN interconnectivity versus change CES-D scores. FIG. 2D shows a graphical representation of changes in DMN-SN interconnectivity versus change in Mental Health Continuum—Short Form (MHC-SF) score.

FIGS. 3A-3B show results for IL-18 levels decreased following the intervention. FIG. 3A shows IL-18 levels decreased by 25% post-intervention (p<0.05, d=−0.64, n=15). FIG. 3B shows the neuroimmune link by reduced IL-18, correlated with decreased rsFC between right amygdala and bilateral precuneus (posterior DMN).

FIGS. 4A-4C show psychological questionnaire results. FIG. 4A shows percentage change from baseline for each group across psychological questionnaires. FIG. 4B shows CES-D and questionnaire scores in the low-intensity follow up. FIG. 4C shows MHC-SF and questionnaire scores in the low-intensity follow up.

FIGS. 5A-5B show immune mediator results. FIG. 5A shows change from baseline for each group across cytokines. FIG. 5B shows cumulative distributions of percentage decrease in cytokines.

FIGS. 6A-6H generally show the association between brain plasticity, peripheral inflammation, and psychological state. FIGS. 6A-6B show the resting-state fMRI interaction effect: Test>Control, Post>Pre-intervention. FIGS. 6C-6E show the association between changes in insula connectivity and reductions in cytokine levels. FIGS. 6F-6H show the association between changes in insula connectivity and improvement in psychological scores.

FIGS. 7A-7C show the improvement of the Insomnia Severity Index (ISI) between groups.

FIG. 8A shows results for depression in Parkinson's disease (PD) patients, post intervention improvement in Beck's Depression Inventory II (BDI-II) scores. This improvement was correlated with brain connectivity changes within the limbic circuit. FIGS. 8B-8D show correlations between increased thalamic AV connectivity and improvement in depressive symptoms.

FIGS. 9A-9B show correlations between increased thalamic AV-mPFC connectivity and self-engagement with the software application of the invention for emotion regulation activities.

FIG. 10 shows a Ven diagram of improved psychological comorbidities, mobility and overall quality of life.

FIG. 11 shows synergism in mechanisms of action of Pimicotinib and the software application of the invention.

FIG. 12A shows a flow chart of a patient medical journey and unmet needs. FIG. 12B shows protocol interventions and related app features for an individual undergoing the methods of the invention.

For simplicity and clarity of illustration, elements shown in the figures are not necessarily drawn to scale, and the dimensions of some elements may be exaggerated relative to other elements. In addition, reference numerals may be repeated among the figures to indicate corresponding or analogous elements.

### DETAILED DESCRIPTION

The present invention seeks to address these unmet needs by harnessing the potential of digital therapeutics (DTx), which are software-based products designed to provide tailored care to patients. The invention is a system and method for treating TGCT, which is comprised of an integrated set of non-pharmacological interventions, which can be implemented independently as a standalone therapy or in a hybrid mode in combination with pharmacological interventions, where it is synchronized with the drug and enhances its efficacy. The method is delivered through a digital platform, such as a smartphone application.

A key innovation of the present invention lies in its recognition of the unique therapeutic window presented by TGCT treatment, particularly when combined with CSF1R inhibitors. Unlike many chronic conditions where treatment merely manages symptoms, TGCT treatment with CSF1R inhibitors is disease-modifying, as the drugs actually shrink the tumors rather than simply controlling symptoms. As the tumor shrinks, patients experience a critical period during which targeted interventions can achieve rapid functional recovery. The present invention capitalizes on this window by providing structured, guided support that goes beyond standard care. During this therapeutic window, the digital interventions described herein are specifically designed to help patients maximize their functional gains. While the pharmaceutical treatment addresses tumor reduction at the cellular level, the coordinated digital interventions simultaneously work to: (1) reduce the systemic inflammation that extends beyond the tumor itself, (2) retrain pain perception and modulation pathways that have been sensitized by chronic disease, (3) restore range of motion and joint function through precisely calibrated physiotherapy, and (4) address the psychological burden that accumulates during the often lengthy diagnostic and treatment journey. This structured support approach transforms the tumor shrinkage period from a passive waiting phase into an active rehabilitation period, enabling patients to achieve functional recovery that parallels or even exceeds the pace of tumor reduction. Furthermore, the invention recognizes that TGCT patients face distinct challenges compared to those with purely symptom-based chronic conditions. Because the disease is rare, patients often lack access to specialized care and feel isolated in their treatment journey. Because the disease is “benign,” its impact on quality of life is frequently underestimated by healthcare providers and support networks. And because effective pharmaceutical treatments are relatively new, there is limited clinical guidance on optimal supportive care during treatment. The present invention addresses all of these gaps by providing evidence-based, comprehensive, accessible interventions that patients can access independently while remaining coordinated with their pharmaceutical treatment regimen.

The methods of the invention are predicated on the integration of any number of five complimentary principal digital modes of action, which are designed to enhance the following peripheral and neural processes, into a single treatment protocol: (1) reducing inflammation, (2) rebalancing emotion & cognitive networks, with emphasis on pain perception and modulation, (3) improving motor function, (4) enhancing brain plasticity, and (5) triggering the reward system.

Regarding inflammation, TGCT involves localized synovial inflammation driven by cytokine overproduction, leading to joint swelling, stiffness, and tissue damage. The invention introduces a digital modality using mindfulness, breathing, and guided imagery to reduce stress, modulate the HPA axis and sympathetic nervous system, and lower cortisol, adrenaline, and pro-inflammatory cytokines. By downregulating systemic inflammation and the RANKL pathway, these interventions help mitigate osteoclast-driven bone and cartilage destruction, preserving joint integrity and function.

Regarding rebalancing Emotion and Cognitive Networks, with Emphasis on Pain Perception and Modulation, cytokines such as TNF-α, IL-1, IL-6, granulocyte-macrophage colony-stimulating factor (GM-CSF), IL-4, IL-10, IL-13, and IL-17, are involved in the modulation of pain.

### Methods of the Invention

In one embodiment the invention provides a method for modulating one or more disease-related markers, symptoms, or disease progression in an individual with benign connective tissue tumor originating from, or causing, dysregulated immune response, the method comprising:
delivering a digitized therapeutic plan to the individual, wherein the digitized therapeutic plan comprises a plurality of therapeutic interventions selected from: at least one physical intervention and at least one psychological intervention;
converting the plurality of therapeutic interventions into a plurality of targeted interaction outputs for presentation to the individual; and
adaptively delivering the digitized therapeutic plan, tracking the individual's responses and associated performance metrics, and continuing until a predetermined condition is met.

In one embodiment the invention provides a method for modulating one or more disease-related markers, symptoms, or disease progression in an individual with benign connective tissue tumor originating from, or causing, dysregulated immune response, the method comprising:
delivering a digitized therapeutic plan to the individual, wherein the digitized therapeutic plan comprises therapeutic interventions selected from: at least one physical intervention and at least one psychological intervention;
converting the therapeutic interventions into a plurality of targeted interaction outputs for presentation to the individual; and
adaptively delivering the digitized therapeutic plan, tracking the individual's responses and associated performance metrics, and continuing until a predetermined condition is met.

As described herein, the therapeutic interventions refer to any number of interventions. In one embodiment the therapeutic interventions comprise at least one physical intervention and at least one psychological intervention, either carried out together, or sequentially. Thus, the terms “therapeutic interventions” and “plurality of therapeutic interventions” can be understood interchangeably when referring to multiple therapeutic interventions.

In one embodiment the method further comprises generating the digitized therapeutic plan for implementation by the individual. In one embodiment the method further comprises determining the digitized therapeutic plan for implementation by the individual.

In one embodiment the digitized therapeutic plan comprises a plurality of therapeutic interventions.

In one embodiment the methods of the invention are for decreasing pro-inflammatory cytokines. Examples of pro-inflammatory cytokines include, but are not limited to: TNF-α, IL-1β, IL-6, IL-12, IL-17, IL-18, IL-23, IFN-γ, GM-CSF, MIF. By reducing inflammation and targeting a primary pathology of the disease, downstream effects such as pain and limited range of motion can be improved, which can then be complemented by therapies specifically aimed at pain management and joint mobilization.

In one embodiment the methods of the invention are for modulating one or more disease-related markers, symptoms, or disease progression in an individual with arthritis. Examples of arthritis include, but are not limited to: osteoarthritis, rheumatoid arthritis, psoriatic arthritis, gout, ankylosing spondylitis, Juvenile Idiopathic Arthritis (JIA), reactive arthritis, enteropathic arthritis.

In one embodiment the methods of the invention are for modulating at least one physiological pathway selected from: the hypothalamic-pituitary-adrenal (HPA) axis, the sympathetic nervous system (SNS), glucocorticoid secretion, adrenergic signaling, cytokine production, or a combination thereof.

In one embodiment the invention provides a method for modulating one or more disease-related markers, symptoms, or disease progression in an individual with benign connective tissue tumor originating from, or causing, dysregulated immune response, the method comprising:
delivering a digitized therapeutic plan to the individual, wherein the digitized therapeutic plan comprises a plurality of therapeutic interventions selected from: at least one physical intervention and at least one psychological intervention; and
converting the plurality of therapeutic interventions into a plurality of targeted interaction outputs for presentation to the individual.

In one embodiment the method further comprises adaptively delivering the digitized therapeutic plan, tracking the individual's responses and associated performance metrics, and continuing until a predetermined condition is met.

As used herein, the term “modulating” refers to changing, adjusting, modifying, disease modification, altering, influencing, or affecting the level, magnitude, pattern, rate, variability, expression, manifestation, severity, or progression of a parameter, condition, symptom, or marker. Modulation may include increasing, decreasing, enhancing, improving, amplifying, reducing, attenuating, suppressing, stabilizing, normalizing, treating, or delaying the onset or progression of such parameter. Modulation may occur in any direction and may represent a partial, complete, transient, sustained, direct, or indirect effect. The modulation may be assessed relative to a baseline, control condition, population norm, or expected natural disease trajectory.

As used herein the term “benign tumor” (also known as a benign neoplasm or benign growth), refers to a noncancerous collection of cells. Unlike cancerous tumors, benign tumors are slow-growing, stay in their primary location and do not spread to local structures or to distant parts of the body. However, a large benign tumor may press on nearby tissues or organs, or induce secondary inflammation in the surrounding tissue. Depending on the extent of such interference, benign tumors may or may not cause symptoms. Regarding connective tissue tumors: connective tissue is biological tissue that is found in between other tissues in the body. Most types of connective tissue consist of three main components: elastic and collagen fibers, ground substance, and cells. Various types of specialized tissues and cells are classified under the spectrum of connective tissue, and are as diverse as brown and white adipose tissue, cartilage and bone, muscle, ligament and articular (joint) tissue. Benign connective tissue tumors include mainly fibromas, lipomas, nerve sheath tumors, hemangiomas, fibrous histiocytoma, and osteomas.

Regarding “tumors originating, or causing, dysregulated immune response”: Cells of the immune system—such as macrophages, mast cells, plasma cells, and eosinophils—are found scattered in loose connective tissue, providing the ground for starting inflammatory and immune responses upon the detection of antigens. Immune dysregulation refers to abnormal immune system functioning, leading to improper responses, and can cause autoimmune diseases, chronic inflammation, and cancer. A dysregulated immune response (e.g., overexpression of the cytokine CSF-1and other pro-inflammatory cytokines) can lead to a massive infiltration of immune cells into the local tissue (e.g., joints). These cells may coalesce to form a solid tumor (these are “tumors originating from dysregulated immune response”). Furthermore, many tumors (regardless of their origin) abnormally secret pro-inflammatory cytokines which cause chronic, improper, and unchecked activation of the immune system (these are “tumors causing dysregulated immune response”). In some cases, like TGCT, the tumors could belong to both of these classes (i.e., they both originate from and cause dysregulated immune response).

As used herein, and in one embodiment, “converting the plurality of therapeutic interventions into a plurality of targeted interaction outputs for presentation to the individual” refers to the transformation of selected therapeutic interventions into user-perceivable stimuli and interactive elements that can be delivered through a digital interface. For example, as a software application on a smartphone. This process involves mapping each intervention type to appropriate output formats based on its functional goals, the capabilities of the user interface, and the individual's preferences or accessibility needs. For example, motor skills or physical interventions may be rendered as animated exercise demonstrations, touch-sensitive targets for coordination tasks, audio prompts for vocal exercises, or haptic feedback for rhythm training. Psychological interventions may be presented as video content for guided imagery, audio narration for mindfulness, or interactive questionnaires for cognitive behavioral therapy. The conversion process ensures that each therapeutic element is delivered in a format that is engaging, accessible, and aligned with the intended therapeutic outcome. In certain embodiments, determining the therapeutic plan may encompass any suitable mode of obtaining or defining the therapeutic plan for the individual. For example, the therapeutic plan may be acquired from an existing clinical protocol, guideline, standardized care pathway, or previously established therapy regimen. In other embodiments, the therapeutic plan may be generated de novo based on patient-specific characteristics, assessments, goals, or clinician input. In one embodiments, the therapeutic plan may be adapted or modified from a pre-existing plan, such as by revising one or more therapeutic elements, dosing schedules, behavioral interventions, or support activities in accordance with patient needs or observed responses. Any combination of acquiring, generating, and adapting a therapeutic plan is also contemplated, and the plan may continue to evolve dynamically over time in response to ongoing monitoring or feedback.

In one embodiment the invention provides a method for modulating one or more disease-related markers, symptoms, or disease progression in an individual with benign connective tissue tumor, the method comprising:
delivering a digitized therapeutic plan to the individual, wherein the digitized therapeutic plan comprises a plurality of therapeutic interventions selected from: at least one physical intervention and at least one psychological intervention;
converting the digitized therapeutic plan into a plurality of targeted interaction outputs for presentation to the individual; and
adaptively delivering the digitized therapeutic plan, tracking the individual's responses and associated performance metrics, and continuing until a predetermined condition is met.

In one embodiment the invention provides a method for modulating one or more disease-related markers, symptoms, or disease progression in an individual with a [symbol] , the method comprising:
delivering a digitized therapeutic plan to the individual, wherein the digitized therapeutic plan comprises a plurality of therapeutic interventions selected from: at least one physical intervention and at least one psychological intervention;
converting the digitized therapeutic plan into a plurality of targeted interaction outputs for presentation to the individual; and
adaptively delivering the digitized therapeutic plan, tracking the individual's responses and associated performance metrics, and continuing until a predetermined condition is met.

In one embodiment the tumor originates from, or causes, dysregulated immune response in the individual.

Although the therapeutic interventions of the invention provide methods of treating, preventing, or alleviating symptoms in an individual affected by various diseases, the methods can also be utilized by someone who is otherwise not affected by these diseases. For example, a person who aims at increasing his/her general wellbeing by improving psychological or motor function. Furthermore, any of the digital therapy interventions, and their corresponding aims can be understood as used to either treat, prevent, alleviate symptoms, or a combination thereof. Therefore “treat, prevent, alleviate symptoms” can be understood as doing each of them separately, or in combination, in various embodiments. Since the methods of the present invention are effective in any of those areas, their capability to treat/prevent/alleviate symptoms, are often considered together.

As used herein, the term “delivering” refers to providing or making the digitized therapeutic plan accessible to an individual by any means, including electronic, digital, or network-based methods, such that the individual can receive, view, or interact with the plan.

In one embodiment the delivering of the therapeutic plan comprises interventions configured to activate at least one of the following therapeutic modes of action: (i) reducing inflammation; (ii) rebalancing emotional and cognitive networks including pain perception and modulation; (iii) improving motor function; (iv) enhancing brain plasticity using sensory inhibition, sensory substitution, or sensory integration; and (v) triggering the reward system.

As used herein the term “digitized therapeutic plan” refers to a therapeutic regimen or intervention that is at least partially implemented, accessed, or executed on a personal electronic device.

As used in this description of the present invention, the terms “drug” or “drug therapy” shall encompass treatment with any medication, whether approved or not by the relevant regulatory authorities (such as the FDA).

As used herein, the individual using or undergoing the therapeutic interventions is referred to in a number of ways. For example, a “user” can also be referred to, and understood interchangeably, as a “patient”, “individual”, “subject”, “participant”, “recipient”.

As used herein, “therapeutic plan” refers to a structured set of therapeutic interventions, strategies, or activities. The therapeutic plan may be personalized based on individual attributes, clinical data, behavioral responses, or performance metrics, and may include physical interventions, psychological interventions, or combinations thereof. It may be delivered digitally, adaptively modified over time, and presented through targeted interaction outputs.

As used herein “psychological” interventions are generally directed towards interventions that affect psychological and/or behavioral aspects of an individual. As such, psychological interventions are generally related to behavioral, social and psychological tasks, as will be described. As used herein “physical” interventions are generally directed towards interventions that improve motor functions in an individual.

As used herein, “adaptively delivering” refers to the continuous and responsive administration of the therapeutic plan to the individual, wherein delivery occurs in a manner that dynamically adjusted over time based on the individual's responses, performance metrics, and evolving therapeutic needs. This includes ongoing presentation of targeted interaction outputs as and when needed throughout the course of the therapeutic plan, with modifications to timing, content, intensity, or modality to optimize therapeutic outcomes and align with predetermined conditions.

As used herein, the term “modality” refers to therapeutic interventions designed to target specific functional domains relevant to the treatment of Parkinson's disease. Modalities include, but are not limited to: physical interventions (e.g., exercises to improve gait, dexterity, or coordination) and psychological interventions (e.g., techniques to support mood regulation, emotional resilience, or stress reduction).

As used herein, “multi-modality” refers to the integration of two or more such therapeutic modalities within a unified treatment plan. By combining interventions across domains, the system enables synergistic effects. This integrative approach supports optimization of therapeutic outcomes, allowing the system to dynamically adjust treatment based on evolving user needs and performance data. Furthermore, when therapeutic interventions are delivered in combination with the administration of medicinal agents, this introduces an additional dimension, or modality, into the integrated treatment plan, enhancing synergism.

In one embodiment the invention provides a method for modulating one or more disease-related markers, symptoms, or disease progression in an individual with Tenosynovial Giant Cell Tumor (TGCT), the method comprising:
delivering a digitized therapeutic plan to the individual, wherein the digitized therapeutic plan comprises a plurality of therapeutic interventions selected from: at least one physical intervention and at least one psychological intervention;
converting the digitized therapeutic plan into a plurality of targeted interaction outputs for presentation to the individual; and
adaptively delivering the digitized therapeutic plan, tracking the individual's responses and associated performance metrics, and continuing until a predetermined condition is met.

In one embodiment the benign connective tissue tumor is Tenosynovial Giant Cell Tumor (TGCT).

In one embodiment the method further comprises obtaining a plurality of attributes associated with the individual from a plurality of data acquisition sources to generate an initial profile for the individual, wherein the digitized therapeutic plan further comprises adaptation based on the initial profile.

In one embodiment the method further comprises obtaining a plurality of attributes associated with the individual from a plurality of data acquisition sources to generate an initial profile for the individual, wherein the determining of the therapeutic plan further comprises adapting the therapeutic plan based on the initial profile. In various embodiments machine learning is employed to assist in any of the following selected from: generating the initial profile, generating the dynamic user profile, adapting the therapeutic plan, adapting the therapeutic intervention, recursive adaptations, Bayesian inference, feedback mechanisms, performance tracking, data analysis, or combinations thereof. In one embodiment the at least one therapeutic intervention comprises real-time adaptive feedback based on sensor input during performance of the intervention.

In various embodiments the term “adaptation” refers to the adjustment, modification, personalization, or tailoring of one or more aspects of the therapeutic plan to the individual. Aspects for adaptation include, but not limited: timing, content, intensity, frequency, or delivery, based on information, attributes, or responses associated with the individual. In various embodiments the adaptation is configured to be optimized for the individual's characteristics, needs, or progress.

In one embodiment adaptation further comprises selecting and implementing therapeutic intervention parameters based on at least one attribute, of the plurality of attributes, of the individual selected from: age, hobbies, music preferences, spoken language, caregiver involvement preferences, disease characteristics, tumor location, tumor size, disease severity, the disease-related markers, the symptoms, pain levels, range of motion limitations, joint stiffness, educational background, professional background, and individual-defined goals.

In one embodiment the plurality of attributes comprises synchronizing multimodal data streams from the plurality of data acquisition sources into a time-aligned dataset, the synchronizing comprising associating each data stream with a unified temporal reference such that data samples, features, or events from the plurality of data acquisition sources correspond to common timestamps or time intervals, thereby enabling identification of temporal relationships and co-occurring patterns across modalities, and wherein the multimodal data streams comprise two or more of: motion data, speech data, gaze-tracking data, physiological signals, touch interaction data, accelerometer data, gyroscope data, pressure sensor data, keyboard and/or touchscreen interaction patterns, facial expression data, autonomic nervous system signals, or response-timing data. As used herein, “multimodal data streams” (as related to “data stream modalities”) refer to multiple types of data collected from different sources, such as motion, speech, touch, and physiological signals, that together provide a comprehensive view of user behavior and state.

In one embodiment the synchronizing comprises timestamping each data point from each data stream modality with a common time reference to enable temporal correlation analysis across data stream modalities and detection of cross-domain patterns indicative of Parkinson's disease progression or therapeutic response. In one embodiment the methods further comprises performing data quality assessment on the multimodal data streams, wherein data quality assessment comprises one or more of: detecting missing data, identifying sensor malfunction, filtering noise, detecting outliers, or validating data integrity. In one embodiment the method further comprises applying data imputation techniques to estimate missing data points based on historical patterns, data from other data stream modalities, or population-level models. In one embodiment the method further comprises integrating data from motor attributes, psychological attributes, cognitive attributes, and pharmacological attributes to generate a unified therapeutic response model that characterizes the individual's multidimensional response to the therapeutic plan. As used herein a “unified therapeutic response model” refers to a comprehensive framework that combines diverse data sources to represent how an individual responds to a therapeutic plan over time. As used herein the term “multidimensional response” refers to the individual's combined behavioral, physiological, cognitive, and emotional changes resulting from therapeutic interventions and medication. In one embodiment the unified therapeutic response model comprises quantified relationships between: motor intervention performance and motor symptom changes; and cognitive function changes; psychological intervention engagement and psychological state changes; pharmacological state and performance across all intervention types; and cross-domain effects wherein interventions in one domain affect outcomes in other domains. As used herein “domain” refers to a distinct functional area targeted by therapeutic interventions, such as motor function, cognitive ability, psychological state, or pharmacological status. In one embodiment the unified therapeutic response model enables holistic assessment of the individual's Parkinson's disease state by capturing interactions and compensatory mechanisms across motor, cognitive, psychological, and pharmacological domains that would not be apparent from assessment of individual domains in isolation.

Examples of data acquisition sources includes, but are not limited to: electronic medical record (EMR) data, clinical practice guidelines, evidence-based protocols, patient input, expert consultation including medical professionals, allied health professionals, and user experience specialists, and machine learning analysis of aggregated patient outcome data to identify and optimize effective therapeutic intervention combinations. Electronic Medical Records (EMR) refers to digital medical records maintained by a healthcare provider for an individual patient. Comprised within that, the EMR data can include any of the following: patient demographics, medical history and prior diagnoses, current and past medications and prescriptions, documented allergies and contraindications, vital signs and other routine clinical measurements, laboratory test results, imaging reports (e.g., X-ray, CT, MRI), clinician notes, treatment plans and care instructions, and records of visits, appointments, and clinical encounters.

In one embodiment the therapeutic plan comprises performing the at least one motor skill intervention concurrently with the at least one psychological intervention. In one embodiment, the digitized therapeutic plan incorporates dual tasking, wherein the individual is engaged in two or more distinct therapeutic interventions simultaneously. Dual tasking may include, for example, performing a physical activity while concurrently engaging in a psychological task.

In one embodiment the therapeutic interventions occur concurrently. In one embodiment the therapeutic interventions occur at least partially concurrently. In one embodiment the therapeutic interventions occur sequentially. For example, one therapeutic intervention may involve a combined physical and psychological intervention, whereas another therapeutic intervention may involve a physical intervention followed by a psychological intervention. Any number of such interventions can be comprised within the digitized therapeutic plan, and in any order. In one embodiment the digitized therapeutic plan comprises performing the at least one physical intervention concurrently with the at least one psychological intervention. In one embodiment the digitized therapeutic plan comprises performing the at least one physical intervention concurrently with the at least one psychological intervention, or sequentially, or a combination thereof. The digitized therapeutic plan can comprise any number of therapeutic interventions. In one embodiment the digitized therapeutic plan comprises between 1 and 1000 therapeutic interventions. In one embodiment the digitized therapeutic plan comprises between 1 and 10,000 therapeutic interventions.

In one embodiment the adaptation further comprises selecting and implementing therapeutic intervention parameters based on at least one attribute of the individual selected from: age, hobbies, music preferences, spoken language, caregiver involvement preferences, disease characteristics, tumor location, tumor size, disease severity, the disease-related markers, the symptoms, pain levels, range of motion limitations, joint stiffness, educational background, professional background, and individual-defined goals.

In one embodiment the therapeutic plan further comprises incorporating at least one personal preference of the individual selected from: age-appropriate content, hobby-related themes, preferred music genres, spoken language, caregiver involvement level, educational background, professional background, or a combination thereof, wherein the therapeutic plan is adapted according to the at least one personal preference. Any of these personal preferences can be input into the systems of the invention for the purpose of updating an individual's profile, updating the therapeutic plan, or a therapeutic intervention itself.

In one embodiment the therapeutic plan further comprises incorporating at least one personal preference of the individual selected from: age-appropriate content, hobby-related themes, preferred music genres, spoken language, caregiver involvement level, educational background, professional background, patient-defined goals, or a combination thereof, wherein the therapeutic plan is adapted according to the at least one personal preference.

Examples of patient-defined goals include, but are not limited to: improving joint mobility, reducing joint pain, decreasing swelling, improving range of motion, enhancing functional use of the affected limb, reducing stiffness, improving balance and stability, preventing joint instability or “giving way,” reducing episodes of joint catching, maintaining or restoring daily activity levels, improving sleep quality, managing fatigue, and managing emotional or psychological well-being, or a combination thereof.

In one embodiment the digitized therapeutic plan is further dynamically adapted based on changes to at least one of the attributes of the individual during the course of the digitized therapeutic plan.

In one embodiment the dynamically adapting of the therapeutic plan comprises applying one or more safety-based pacing rules that adjust or limit physical interventions based on real-time or periodic measurements of joint stiffness, swelling, pain levels, or range of motion, to prevent over-exertion, flare-ups, or unsafe loading of the affected joint and to support responsible functional recovery.

In one embodiment, the safety-based pacing rules are individualized based on each patient's symptom patterns and response history. The system learns from each patient's data, identifying their personal indicators of impending flare-ups and their typical recovery patterns. Over time, this enables increasingly accurate predictions of which days will be suitable for more aggressive rehabilitation versus days requiring gentler approaches. The safety-based pacing rules represent a significant advantage of the digital therapeutic system over traditional paper-based or video-based exercise prescriptions, which cannot adapt to the patient's changing status. By dynamically balancing the competing goals of maximizing functional gains while minimizing risks of over-exertion and injury, the pacing system supports responsible functional recovery that is optimized for each patient's individual disease trajectory and response pattern.

In one embodiment the method further comprises:
dynamically monitoring the individual's monitored parameters during delivery of the digitized therapeutic plan;
generating a dynamic user profile based on the initial profile and monitored parameters of the individual selected from: Electronic Medical Records (EMR) data, motor attributes, psychological attributes, range of motion measurements, pain levels, joint function, pharmacological attributes, tumor response parameters, disease progression, feedback of the individual, feedback of a medical practitioner, drug intake parameters, or a combination thereof; and
adapting the digitized therapeutic plan based on the dynamic user profile.

Other monitored parameters can include, but are not limited to: symptom improvements and functional recovery progression. In one embodiment the method further comprises recursively adapting the digitized therapeutic plan based on updates to the dynamic user profile, the performance metrics, and the individual's responses.

As used “recursively adapting” refers to an iterative and ongoing process of modifying the digitized therapeutic plan based on updated information, including changes to the dynamic user profile, performance metrics, and individual responses, and the likes.

In one embodiment the recursive adapting of the therapeutic plan comprises dynamically identifying and modifying intervention parameters to implement optimized therapeutic outcomes towards a predetermined therapeutic goal. In embodiment the predetermined therapeutic goal is the same as the predetermined condition. Examples of predetermined therapeutic goals include, but are not limited to: improving joint mobility, reducing joint pain, decreasing swelling, enhancing range of motion, preventing or minimizing joint stiffness, improving functional use of the affected limb, reducing episodes of joint instability or “giving way,” minimizing mechanical symptoms such as joint catching, maintaining or increasing daily activity levels, preserving or restoring independence in motor tasks, optimizing recovery after surgical intervention, managing fatigue, improving sleep quality, enhancing participation in social or recreational activities, and achieving a target functional score on a standardized musculoskeletal or quality-of-life assessment, etc.

In one embodiment the recursive adapting of the digitized therapeutic plan comprises dynamically identifying and modifying the therapeutic intervention parameters to implement optimized therapeutic outcomes towards a predetermined therapeutic goal.

In one embodiment the therapeutic intervention parameters comprise any of the following selected from: type, timing, dosage, intensity, sequence, duration, frequency, mode of delivery, level of guidance, feedback modality, language adaptation, complexity level, interface configuration, integration with external systems, or a combination thereof, of one or more of the therapeutic interventions.

Examples of ‘type’ include, but are not limited to: type of therapeutic intervention, type of medication, type of physical exercise. Examples of ‘timing’ include, but are not limited to: time of day of intervention, timing relative to symptom onset, timing relative to medication administration, timing relative to patient-reported symptoms. Examples of ‘dosage’ include, but are not limited to: amount of medication, number of repetitions in an exercise session, number of tasks per session. Examples of ‘intensity’ include, but are not limited to: resistance level in a physical exercise, level of psychological challenge in a task, vibration amplitude in haptic feedback, degree of stretching or mobilization. Examples of ‘sequence’ include, but are not limited to: order of exercises, order of administered medications, sequence of multi-modal interventions, sequence of joint mobilization steps. Examples of ‘duration’ include, but are not limited to: length of each therapy session, duration of medication course, duration of continuous monitoring, duration of a biofeedback session. Examples of ‘frequency’ include, but are not limited to: number of sessions per week, frequency of medication administration, frequency of symptom reporting, frequency of device notifications. Examples of ‘mode of delivery’ include, but are not limited to: in-person, remote/digital delivery, virtual reality-based delivery, augmented reality-guided delivery. Examples of ‘level of guidance’ include, but are not limited to:
fully guided intervention, self-directed intervention, therapist-assisted intervention, automated adaptive guidance. Examples of ‘feedback modality’ include, but are not limited to: visual feedback, auditory feedback, haptic feedback, real-time performance analytics. Examples of ‘language adaptation’ include, but are not limited to: different spoken languages, different written language formats, culturally adapted instructions, simplified versus technical terminology. Examples of ‘complexity level’ include, but are not limited to: simplified instructions, advanced task difficulty, multi-step cognitive tasks, multi-joint exercise coordination. Examples of ‘interface configuration’ include, but are not limited to: touchscreen interface, gesture-controlled interface, voice-controlled interface, multi-sensor integrated interface. Examples of ‘integration with external systems’ include, but are not limited to: wearable activity tracker, electronic health record system, home monitoring devices, cloud-based analytics platforms.

In one embodiment the recursive adapting is performed using a feedback control algorithm, Bayesian inference, or a combination thereof.

In one embodiment the at least one physical intervention is selected from: physiotherapy, range-of-motion exercises, mobility training, limb agility exercises, functional movement training, balance exercises, proprioception exercises, postural stability training, strength training, stretching exercises, coordination training, low impact training, aerobics exercises, flexibility training, and walking, or a combination thereof.

In one embodiment the at least one psychological intervention is selected from: guided imagery, psychoeducation, psychotherapy, cognitive behavioral therapy, stress and anxiety management training, mindfulness-based interventions, body scanning training, sleep hygiene, fatigue training, pain psychology, pacing training, acceptance and commitment therapy (ACT), dialectical behavior therapy (DBT), attention training techniques, psychodynamic therapy, solution-focused brief therapy (SFBT), narrative therapy, pain therapy, addiction therapy, gestalt therapy, behavioral activation therapy, adjustment therapy, grief therapy, motivational therapy, meaning-centered therapy, creative therapy, expressive therapy, logotherapy, telepsychiatry, and teletherapy, or a combination thereof.

In one embodiment the method further comprises pairing a conditioned stimulus with at least one therapeutic intervention such that subsequent presentation of the conditioned stimulus elicits a therapeutic response associated with the intervention.

In some embodiments the conditioning stimulus is selected from: visual, auditory, tactile, gustatory, olfactory, proprioceptive, and vestibular, or a combination thereof. As used herein “conditioning” (or “conditioning stimulus”) refers to the process of associating a particular cue or stimulus with the administration of a drug or medication, or any other type of intervention (e.g., digitized therapeutic intervention), in order to promote positive effects or outcomes. In one embodiment the conditioning stimulus is delivered before, during, after, or a combination thereof, the administering of the at least one medicinal agent, at least one of the therapeutic interventions, or a combination thereof. When therapeutic interventions are delivered in conjunction with the administration of medicinal agents, the combination may produce a priming effect, wherein the therapeutic activity sensitizes or prepares the individual's physiological or cognitive systems to respond more effectively to the pharmacological treatment, thereby enhancing overall therapeutic efficacy

In certain embodiments, the conditioning stimulus is used to reinforce associative learning between a sensory cue and the therapeutic or pharmacological effect when drug administration is involved. Otherwise, the conditioning stimulus can also be used in combination with the stand alone digitized therapeutic interventions themselves. For example, a distinct light pulse, vibration, or tone may be paired with drug administration or with the initiation of a digital intervention, enabling conditioned therapeutic responses and supporting neurobehavioral reinforcement even in the absence of the active stimulus. This associative pairing enhances expectancy, adherence, and long-term consolidation of treatment gains.

Examples of quality of life factors include, but are not limited to: pain levels, joint function, range of motion, mobility, ability to perform activities of daily living, independence in self-care, participation in work or school, engagement in social or recreational activities, emotional well-being, sleep quality, fatigue levels, ability to travel or commute, psychological stress, anxiety related to disease or treatment, and overall satisfaction with physical health and functional status.

In one embodiment the methods further comprise implementing a nutrition regimen in the individual. In one embodiment the nutrition regimen comprises any of the following selected from: portion sizing to maintain healthy body weight, meal timing, anti-inflammatory diet, joint-supportive nutrients, weight management support, reduction of pro-inflammatory food intake, hydration, Mediterranean diet, or a combination thereof. This embodiment is predicated on the understanding of the important role nutrition plays in supporting recovery and managing inflammation in TGCT patients. The nutrition support component is designed to complement the other interventions and optimize overall health outcomes. For example, given that TGCT is characterized by chronic inflammation, dietary approaches that reduce systemic inflammation may support the other anti-inflammatory mechanisms of the invention (such as HPA axis modulation through stress reduction). Similarly, certain nutrients may specifically support joint health, potentially complementing the drug-induced tumor reduction and the exercise-based rehabilitation. Regarding weight management support, for patients with lower-extremity TGCT, maintaining a healthy body weight reduces mechanical stress on affected joints. The timing of meals may impact energy levels, sleep quality, and the ability to engage in physical interventions. Adequate hydration is important for joint lubrication, overall physiological function, and potentially for managing medication side effects.

In one embodiment the disease-related markers comprise any of the following selected from: tumor size, tumor spread, objective response rate (ORR), inflammation, macrophage recruitment or activity, cytokine levels, angiogenesis, tumor cell proliferation, fibrosis, joint swelling, pain, stiffness, physical function, range of motion, quality of life, patient perception of disease, psychological endpoints, grip strength, and gait stability.

In various embodiments, the primary endpoints of the methods of the invention include, but are not limited to: tumor size, tumor spread, ORR (objective response rate), etc. Examples of secondary endpoints include, but are not limited to: pain, stiffness, physical function, range of motion, quality of life (global impression of change), patient perception of disease, psychological endpoints, swelling, etc.

In one embodiment the benign connective tissue tumor is selected from: TGCT, lipoma, angiolipoma, fibrolipoma, spindle cell lipoma, pleomorphic lipoma, hibernoma, fibroma, desmoid tumor, nodular fasciitis, palmar fibromatosis, plantar fibromatosis, elastofibroma dorsi, myofibroma, leiomyoma, angioleiomyoma, rhabdomyoma, schwannoma, neurofibroma, perineurioma, traumatic neuroma, hemangioma, lymphangioma, glomus tumor, myxoma, intramuscular myxoma, granular cell tumor, solitary fibrous tumor (benign type), Giant Cell Tumor of Bone (GCTB), histiocytic disorders, lipoma arborescens, and synovial chondromatosis

### Combination With Medicinal Agents

As explained above, digital interventions of the types explored herein can be used on a stand-alone basis or in combination with at least one medicinal agent. Generally, an adjunctive medication is a supplementary therapeutic agent used in conjunction with primary treatment to optimize efficacy or address specific aspects of a medical condition, often enhancing overall therapeutic outcomes. As will become clear, any therapeutic agent that achieves this goal in a combination with the digital therapy of the invention is considered within the scope of the invention.

When the digitized therapeutic interventions are combined with medicinal agents (e.g., for TGCT), these digital interventions demonstrate potential to enhance therapeutic efficacy. The combination treatment protocol delivers a holistic therapeutic regimen that synchronizes pharmaceutical effects with digital interventions, effectively creating a “hybrid drug”. This protocol is specifically designed to capture and maximize the therapeutic synergy between drug treatments for benign connective tissue tumors, such as TGCT, and non-pharmaceutical interventions. Among other things, the system and methods includes a scheduling module that generates medication reminders (according to each patient's dosage and timing), to ensure an optimal integration of therapeutic interactions with medication administration. Along with other aspects, this feature of the hybrid drug approach supports adherence to the therapy. By employing a multi-modal approach, with interventions focusing on inflammation and chronic pain combined with physical exercises targeting motor function, alongside the drug treatment, the invention improves both clinical and psychological outcomes in TGCT patients beyond the current standard-of-care.

The combination represents a true “hybrid drug” approach wherein pharmaceutical and digital interventions are deliberately synchronized to create synergistic therapeutic effects that exceed the sum of their individual contributions. The synergy operates through complementary mechanisms of action. CSF1R inhibitors, as a particular example, work “upstream” in the pathological cascade by blocking the receptor that mediates macrophage recruitment and tumor cell proliferation. However, by the time patients initiate CSF1R inhibitor therapy, downstream inflammatory processes are already well-established: pro-inflammatory cytokines are elevated, pain sensitization has occurred, joint damage may be present, and compensatory movement patterns have developed. The digital interventions described herein specifically target these downstream consequences. Critically, the timing and dosing of digital interventions can be coordinated with the pharmaceutical treatment schedule. This temporal coordination is important because: (1) certain digital interventions (such as stress reduction and sleep optimization) may enhance drug absorption and efficacy, (2) the digital interventions can help manage side effects of CSF1R inhibitors, thereby improving adherence, (3) as the drug begins to reduce tumor size, the coordinated physical interventions can capitalize on improved joint space and reduced mechanical symptoms, and (4) psychological interventions can address the anxiety and uncertainty that often accompany pharmaceutical treatment, particularly given the relatively recent introduction of these medications. Moreover, the system continuously monitors both pharmaceutical and digital intervention responses, enabling dynamic adjustment of both components. For example, if monitoring indicates that a patient is responding particularly well to the CSF1R inhibitor with rapid tumor shrinkage, the system can accelerate the intensity of physical rehabilitation interventions to maximize functional gains during this favorable period. Conversely, if a patient experiences side effects from the pharmaceutical treatment, the digital intervention protocol can be adjusted to provide additional supportive care, thereby maintaining adherence to the overall treatment regimen. This is particularly important in TGCT, where treatment duration may extend for many months or even years, and where the disease's “benign” designation may lead some patients to underestimate the importance of consistent treatment adherence.

As used herein the term “combined” (or “combined method”) describes any intervention that includes at least one digital therapy intervention and at least one non-digital therapy intervention e.g., a medicinal agent.

In various embodiments, and as used herein, the terms “drug”, “medication”, “medicinal agent”, “pharmaceutical”, “medicine” or the likes are to be understood interchangeably. Namely, these refer to any substance used to diagnose, prevent, treat, or alleviate symptoms of a medical condition. They can also refer to substances that are generally aimed at maintaining or improving an individual's well-being without specifically targeting a disease. Thus, in various embodiments, the ‘medicinal agent’ can refer to a nutritional supplement, as will be detailed. As used herein, and in various embodiments, the “treatment” of any disease includes any intervention that alleviates symptoms or causes at least one marker of that disease to improve. Therefore, “treatment”, as understood herein, also refers to delayed onset or prevention of a disease, in various embodiments.

In one embodiment the method further comprises administering at least one medicinal agent before, during, after, or a combination thereof, the implementation of at least one of the therapeutic interventions.

In one embodiment the at least one medicinal agent comprises any of the following selected from: CSF1R inhibitor, anti-inflammatory drug, immunomodulatory drug, and cytokine-targeting biologic, or a combination thereof. In one embodiment the at least one medicinal agent comprises any of the following selected from: CSF1R-inhibitor, anti-inflammatory drug, immunomodulatory drug, cytokine-targeting biologic, nirogacestat, denosumab, everolimus, methotrexate, and corticosteroids or a combination thereof. In one embodiment the at least one medicinal agent is a monoclonal antibody.

A “CSF1R inhibitor” refers to any compound, molecule, or agent that partially or fully blocks, suppresses, or modulates the activity, signaling, or expression of the colony-stimulating factor 1 receptor (CSF1R).

In one embodiment the CSF1R-inhibitors comprises any of the following selected from: pimicotinib, pexidartinib, vimseltinib, cabiralizumab or a combination thereof. In one embodiment the CSF1-inhibitors are selected from the group consisting of: pimicotinib, pexidartinib, vimseltinib, cabiralizumab or a combination thereof.

In one embodiment the CSF1R-inhibitors comprise any of the following selected from: Pimicotinib, Pexidartinib, Vimseltinib, BLZ945 (Sotuletinib), JNJ-40346527 (Edicotinib), IACS-9439, BPR1R024, ABT-869 (linifanib), Imatinib, AG013736 (axitinib), SU11248 (sunitinib), BAY 43-9006 (sorafenib), CHIR258 (tandutinib), Ki20227, Emactuzumab (RG7155), Lacnotuzumab (MCS110), Cabiralizumab (FPA008), PD-036032, ARRY-382 (PF-07265804), EI-1071, TE-952 (JTE-952), PLX5622, GW2580, IMC-CS4 (LY3022855), and AMG820 or a combination thereof. In one embodiment Imatinib is multitargeted with CSF1R activity.

In one embodiment the at least one medicinal agent comprises any of the following selected from: CSF1R-inhibitors, nirogacestat, imatinib, sorafenib, denosumab, everolimus, methotrexate, and corticosteroids, or a combination thereof.

Examples of CSF1-inhibitors include, but are not limited to: Pexidartinib, Vimseltinib, Pimicotinib, Cabiralizumab, etc. Examples of medicinal agents for desmoid tumors include, but are not limited to: Nirogacestat, imatinib, sorafenib, pazopanib, AL102, etc. Examples of medicinal agents for Gastrointestinal Stromal Tumor include, but are not limited to: Imatinib, Sunitinib, Regorafenib, Ripretinib, Avapritinib, Bezuclastinib, etc. Examples of medicinal agents for giant cell tumors include, but are not limited to: denosumab. Examples of medicinal agents for subependymal giant cell astrocytoma includes, but is not limited to: Everolimus, Other examples of medicinal agents include: methotrexate, corticosteroids.

In one embodiment the method further comprises the administration of an adjunct agent comprising any of the following selected from: nonsteroidal anti-inflammatory drugs (NSAIDS), immunomodulatory agent, cytokine-targeting biologic, corticosteroids, analgesics, anesthetic agents, or a combination thereof. In one embodiment the anesthetic is for use in pain management.

In one embodiment the combination method, which includes administering at least one medicinal agent, further comprises:
obtaining a plurality of attributes associated with the individual from a plurality of data acquisition sources to generate an initial profile for the individual, wherein the digitized therapeutic plan further comprises adaptation based on the initial profile;
wherein the adaptation further comprises selecting and implementing therapeutic intervention parameters based on at least one attribute of the individual selected from: age, hobbies, music preferences, spoken language, caregiver involvement preferences, disease characteristics, tumor location, tumor size, disease severity, disease-related markers, symptoms, pain levels, range of motion limitations, joint stiffness, educational background, professional background, medicinal agent intake parameters, and individual-defined goals.

In one embodiment the method further comprises a conditioning stimulus delivered before, during, after, or a combination thereof, the administering of the at least one medicinal agent, at least one of the therapeutic interventions, or a combination thereof.

### Systems of the Invention

In various embodiments the systems of the invention are set to continuously gather data through an integrated network of wearables and sensors. These devices monitor vital health metrics including heart rate and rhythm, temperature, respiration, glucose levels, muscle tension, and brain activity, alongside physical movement tracking (e.g., steps). In addition, during in-app interactions, the system collects an array of clinical indicators, including physical characteristics such as joint range of motion and stiffness, pain levels, assessment of physiotherapy exercises, tapping assessment, and emotional state indicators through facial expression analysis. This is complemented by the collection of valuable implicit data from users'daily device usage patterns. This includes behavioral metrics such as typing speed and frequency, communication patterns through phone calls, and overall digital engagement levels. For users with compatible wearable devices, this dataset can be enhanced with advanced biometric information including detailed sleep patterns and gait analysis.

Drawing on both objective measurements of these types and patient-reported outcomes, the platform employs AI-driven analytics to process this comprehensive data across three key dimensions: 1) Tracking and Monitoring: The system continuously analyses symptoms and disease progression, identifying meaningful trends and pattern changes that might indicate important clinical shifts. 2) Treatment Optimization: Using sophisticated algorithms, the system dynamically adjusts its therapeutic approach, calibrating intervention strategies to each user's needs and delivering personalized recommendations for optimal outcomes. 3) Healthcare Provider Integration: All collected data can be exported and shared with healthcare professionals, enabling informed decision-making, or collected for future studies.

This multi-faceted approach to data collection and analysis creates a learning system that not only refines individual treatment protocols but also enhances its overall therapeutic effectiveness. By maintaining detailed records of patient progression through their intervention plan and continuously monitoring physical activity patterns, the platform builds a holistic view of patient health and engagement that enables increasingly precise and effective treatment interventions.

Regarding treatment optimization, there are many levers that can be personalized and adjusted dynamically within the framework of the protocol, based on information supplied by the user as well as data collected by the system, both during onboarding and throughout the user's interactions with the system. Personalization and dynamic adjustment can apply to individual interventions. For example, based on the patient's historical range of motion (ROM) measurement and activity levels, the treatment protocol can adapt the daily activity. The changes to the daily activity may include or preclude specific physiotherapy and adapt the exercises themselves to the patient's ability for that day. In addition, personalization and dynamic adaptation to patients'evolving needs and progress can also be done on the macro level i.e., in regard to the ratio between different modes of action and the way different interventions are integrated into a single treatment protocol.

In one embodiment the invention provides a digital therapeutic system for modulating one or more disease-related markers, symptoms, or disease progression in an individual with benign connective tissue tumor originating from, or causing, dysregulated immune response, the system comprising:
a data-input module configured to onboard the individual and dynamically monitor and store data related to the individual throughout a digitized therapeutic plan; and
a digital therapy delivery module comprising a processor configured to deliver the digitized therapeutic plan comprising a plurality of therapeutic interventions selected from: at least one physical intervention and at least one psychological intervention;
wherein the digital therapy delivery module is further configured to convert the digitized therapeutic plan into a plurality of targeted interaction outputs for
presentation to the individual and track the individual's responses.

In one embodiment the invention provides a digital therapeutic system for modulating one or more disease-related markers, symptoms, or disease progression in an individual with benign connective tissue tumor originating from, or causing, dysregulated immune response, the system comprising:
a data-input module configured to onboard the individual and dynamically monitor and store data related to the individual throughout a digitized therapeutic plan; and
a digital therapy delivery module comprising a processor configured to generate the digitized therapeutic plan comprising a plurality of therapeutic interventions selected from: at least one physical intervention and at least one psychological intervention;
wherein the digital therapy delivery module is further configured to convert the digitized therapeutic plan into a plurality of targeted interaction outputs for presentation to the individual and track the individual's responses.

In one embodiment the digital therapy delivery module comprises software executable on a personal electronic device. In one embodiment the software is in the form of an application for a personal electronic device. In one embodiment the processor is further configured to generate the therapeutic plan. As used herein the term “data-input module” refers to any hardware, software, or combination thereof configured to receive, collect, record, or otherwise capture information or data associated with the individual. The data-input module may include interfaces for manual entry, automated sensors, wearable devices, mobile applications, or other electronic or digital sources, and is capable of onboarding the individual, continuously or intermittently acquiring data, and storing the information for use in generating, adapting, or monitoring a digitized therapeutic plan.

In one embodiment the data-input module is configured to receive data from a plurality of data acquisition sources, wherein the data comprises one or more of: Electronic Medical Records (EMR) data, motion sensor data, position sensor data, environmental sensors data, audio sensors data, location sensors data, audio recording data, speech recognition data, gaze-tracking data, touch interaction data, physiological sensor data, optical sensor, imaging sensors, self-report data, or a combination thereof. In one embodiment the data-acquisition is in real-time, or substantially in real-time.

As used herein the term “digital therapy delivery module” refers to any hardware, software, or combination thereof configured to implement, present, or provide a digitized therapeutic plan to an individual. The module may generate, schedule, adapt, or otherwise manage one or more therapeutic interventions, including physical, psychological, behavioral, and may deliver the interventions via electronic, digital, network-based, or device-mediated means, such that the individual can access, interact with, or execute the therapeutic plan.

In one embodiment the digital therapy delivery module comprises any of the following selected from: a personal electronic device, a graphical user interface, audio interface, haptic interface, or a combination thereof.

As used herein, the term “personal electronic device” is understood to be any electronic device used by a user to carry out any part of the digital intervention. Thus, although it is often a ‘personal’ device, it is also understood to include devices that are not directly owned by the user, but those that are used/synced for the user to implement the digital intervention. The following are examples of personal electronic device, but are not limited to: smartphones, tablets, wearable device, smart TVs, computers, laptops, E-readers, gaming consoles, smartwatches, fitness trackers, portable media players, digital cameras, virtual reality (VR) headsets, augmented reality (AR) device, portable GPS devices, portable Bluetooth devices, portable digital assistant, smart glasses and audio device or any combinations thereof. In various embodiments the personal electronic device is a mobile computing device.

In one embodiment the processor is further configured to:
dynamically monitor any of the following factors of the individual selected from: Electronic Medical Records (EMR) data, motor attributes, psychological attributes, cognitive attributes, pharmacological attributes, and drug intake parameters, or a combination thereof, throughout the implementation of the digitized therapeutic plan;
generate a dynamic user profile, and recursively adapt the digitized therapeutic plan based on the dynamic user profile, performance metrics, and the individual's responses;
or a combination thereof.

In one embodiment the processor is further configured to dynamically monitor any of the following factors of the individual selected from: Electronic Medical Records (EMR) data, motor attributes, psychological attributes, cognitive attributes, pharmacological attributes, and drug intake parameters, or a combination thereof, throughout the implementation of the therapeutic plan, and generate a dynamic user profile.

In one embodiment the processor is further configured to recursively adapt the therapeutic plan based on the dynamic user profile, performance metrics, and the individual's responses.

The digital therapy delivery module may, in certain embodiments, not only execute but also generate or update the therapeutic plan based on data received from the data-input module. Both modules may communicate bidirectionally via secure, encrypted channels to ensure real-time synchronization and data integrity. The data-input module may preprocess sensor or patient-reported data, identify anomalies, and update the dynamic patient profile accordingly. The system may store anonymized session data locally or in cloud infrastructure for longitudinal analysis, algorithm refinement, or regulatory compliance. In one embodiment, the data-input module is configured to receive and synchronize multimodal data streams from multiple data acquisition sources in real-time or near-real-time, wherein the multimodal data streams comprise two or more of: motion sensor data, joint range-of-motion measurements, grip strength data, pain or symptom self-report data, touch or pressure interaction data, imaging-derived metrics, or physiological sensor data. In one embodiment, the processor is further configured to apply time-alignment algorithms to synchronize data streams with different sampling rates into a unified temporal framework. In one embodiment, the processor is further configured to generate a unified therapeutic response model by integrating performance, symptom, and functional data across motor, musculoskeletal, pain, and psychological domains, and wherein the processor uses the unified therapeutic response model to optimize the therapeutic plan. In one embodiment, the unified therapeutic response model comprises a computational model that represents interdependencies between different therapeutic domains using one or more techniques selected from: correlation matrices, regression models, neural network architectures, Bayesian networks, state-space models, or dynamic systems models.

In one embodiment the system further comprises at least one additional device operatively coupled to the digital therapy delivery module, selected from: health monitoring systems, medical devices, haptic devices, external speakers, headphones, virtual reality headsets, augmented reality glasses or devices, biofeedback sensors, wearable activity trackers, smartphones, tablets, smartwatches, personal computational devices, smart speakers, voice assistants, motion tracking sensors, virtual assistant systems, internet hubs, gait sensors, eye-tracking devices, voice recording devices, and wearable devices, or combinations thereof. In various embodiments, the at least one additional device may be operatively coupled to any other device within the system, individually or in combination, to facilitate data exchange, enhance monitoring or feedback, and support the generation, adaptation, and delivery of the digitized therapeutic plan.

As understood herein “software application” is a program that runs on a device. In various embodiment the “software application” is a computer program which performs specific tasks or functions on a device. In embodiment the software application is an app on a smartphone.

In some embodiments, the digital therapeutic system is further configured to comprise gamification elements. For example, gamification elements are configured to enhance user engagement, motivation, and adherence to the therapeutic plan. These elements may include the awarding of digital badges, points, levels, or other forms of recognition based on the individual's progress, consistency, or achievement of therapeutic milestones. The system may also provide rewards or incentives, such as unlocking new content, personalized feedback, or virtual achievements. Additionally, the system may include features for sharing progress with caregivers, clinicians, or peer support networks, and may support synchronization across multiple devices or platforms to ensure continuity of experience and data integrity.

### EXAMPLES

### Example 1 Clinical Results for the Digital Interventions of the Invention

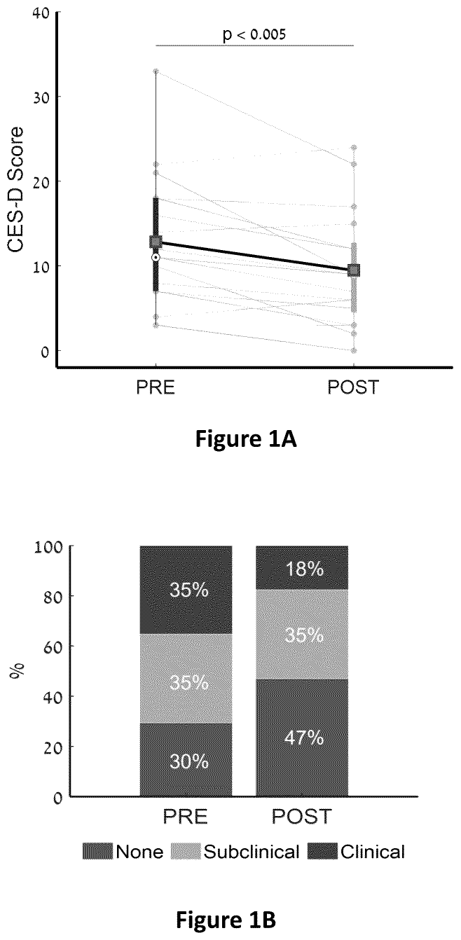

FIG. 1A shows significant reduction in depression scores post-intervention: CES-D scores dropped by 26.6%(p=0.004, d=−0.829). FIG. 1B shows 18% fewer participants met the depression cut-off; 17% more classified as “no depression.”

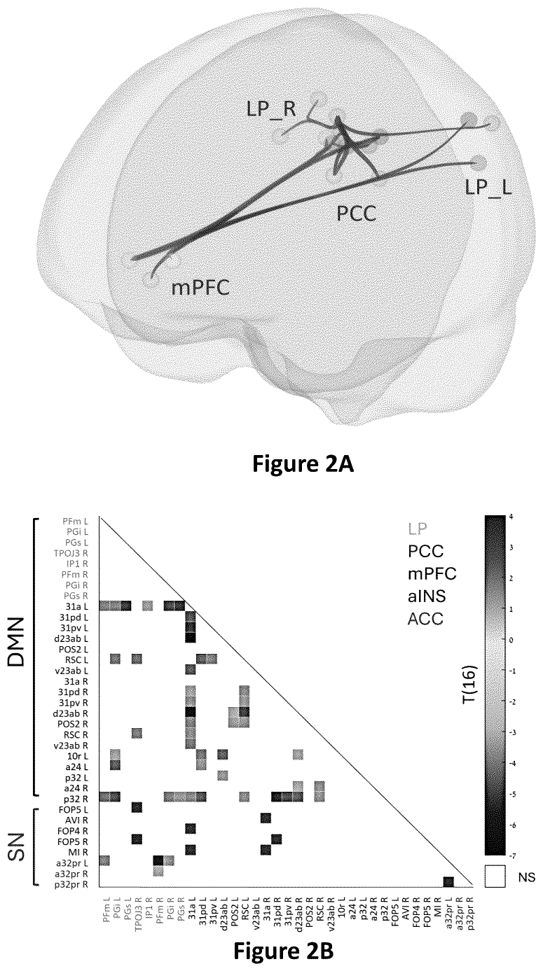

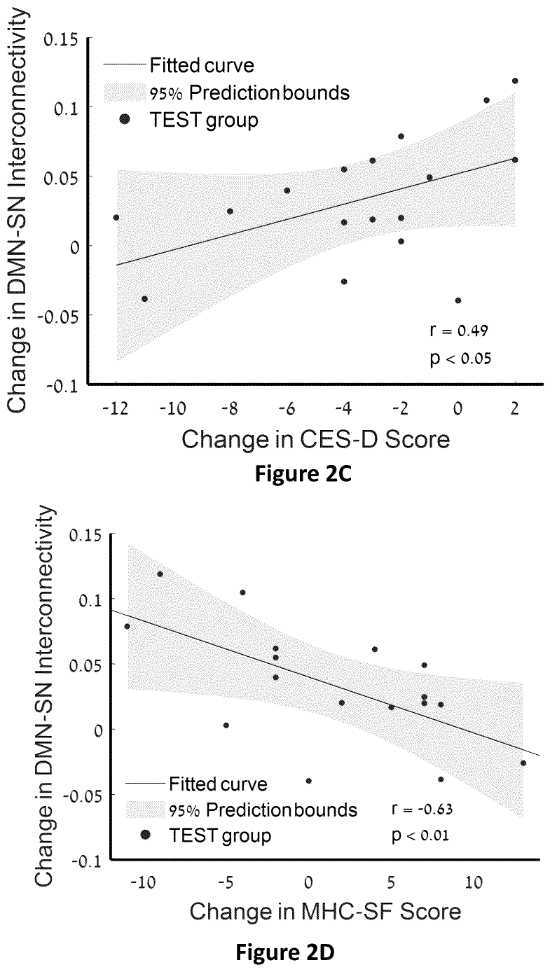

FIGS. 2A-2D show brain cognitivity analysis. Post-intervention changes in DMN: Significant alterations in intra-network connectivity, and clinical correlation: Greater depression score improvement was associated with increased negative DMN-SN rsFC. The DMN is a global brain network, which shows higher activity during rest, and is involved with self-referential processes, recalling memories and daydreaming. Increased rsFC within the DMN is associated with psychological processes and may reflect several clinical aspects of depression, such as rumination, repetitive negative thinking, and unforgiveness. In our study, we demonstrated post-intervention reduction in the intrinsic connectivity within the DMN, specifically, a reduced connectivity between the PCC node and mPFC and LP nodes. This observation aligns with previous studies in late life depression that demonstrated increased connectivity, compared to non-depressed subjects, between the PCC and the anterior DMN, mPFC and sgACC. This reduced connectivity may explain the improvement in depression score, as the connectivity between the anterior and posterior DMN regions has been suggested to reflect positive treatment effects.

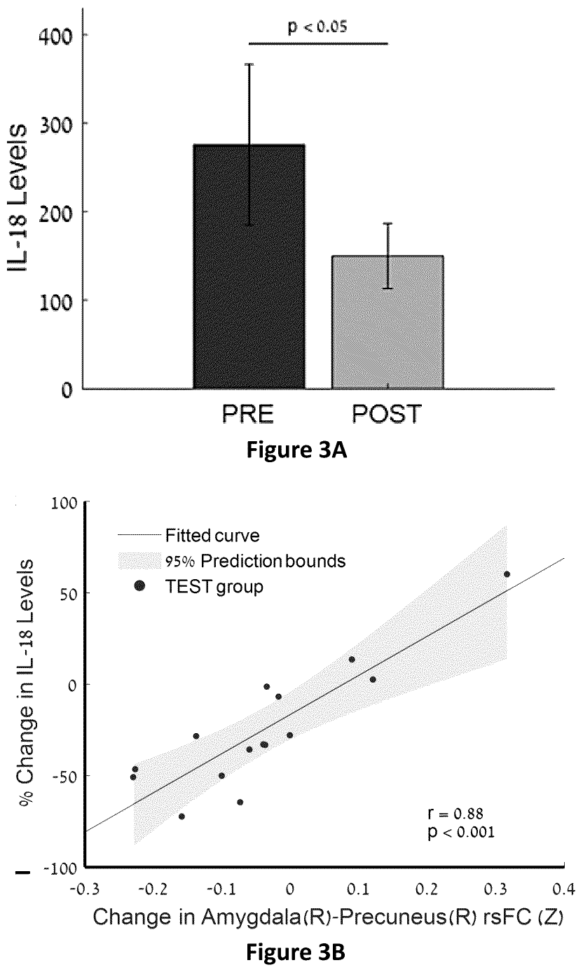

FIGS. 3A-3B show IL-18 levels decreased by 25% post-intervention (p<0.05, d=−0.64, n=15) and neuroimmune link: Reduced IL-18 was correlated with decreased rsFC between right amygdala and bilateral precuneus (posterior DMN). Thus the digital interventions of the invention resulted in reduced depressive symptoms and decreased inflammation marker, correlated with changes in brain connectivity.

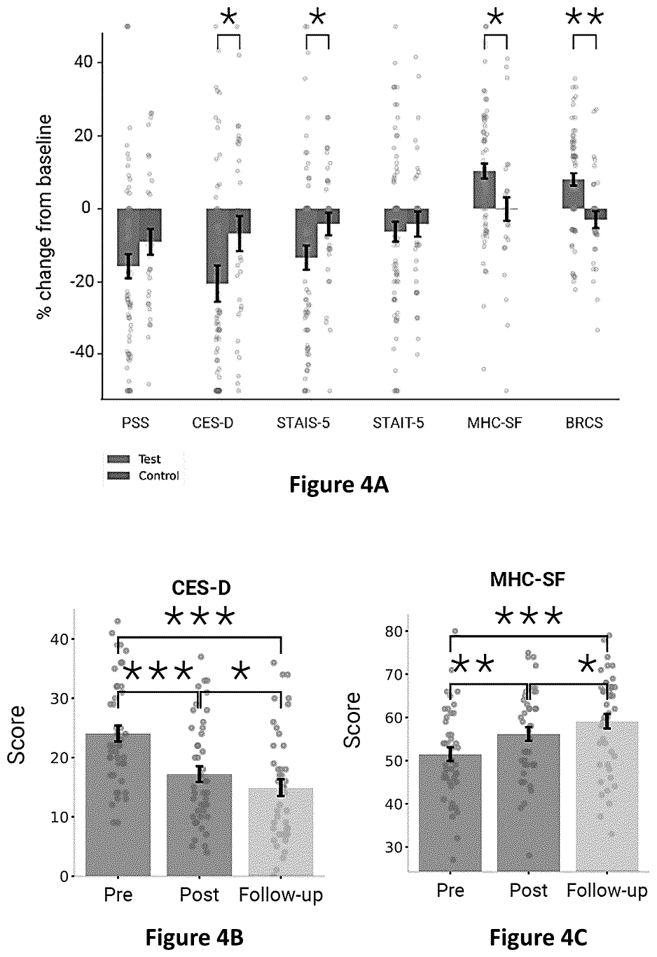

FIGS. 4A-4C show significant improvements observed in the intervention group across key psychological domains and that these positive outcomes were sustained or even enhanced following a 3-week low-intensity follow-up in some domains.

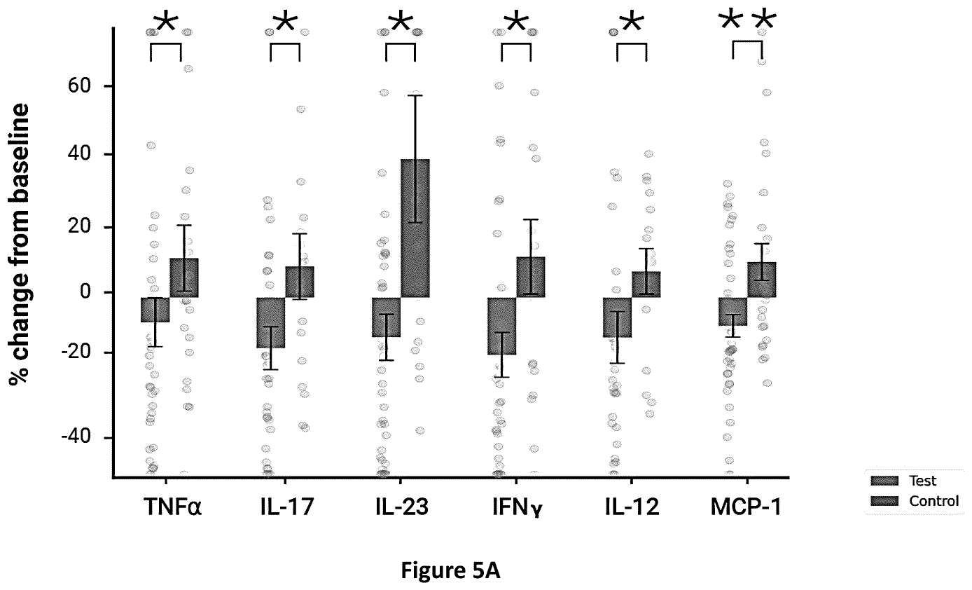

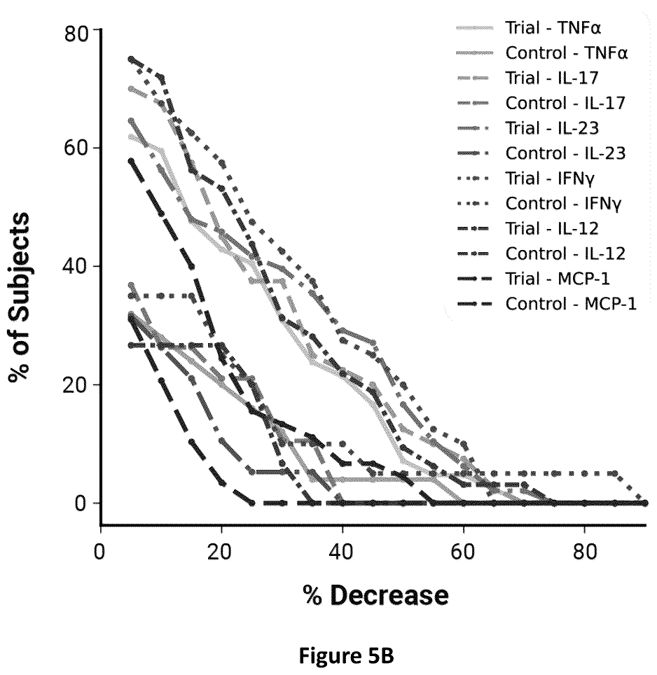

FIGS. 5A-5B show immune mediator results. Significant group-by-time effects were observed for multiple cytokines: TNF-α, IL-17, IL-23, MCP-1, IFN-γ, and IL-12. Furthermore, greater reductions (>5%) in these cytokines were more common in the intervention group, with significant differences vs. control across all markers.

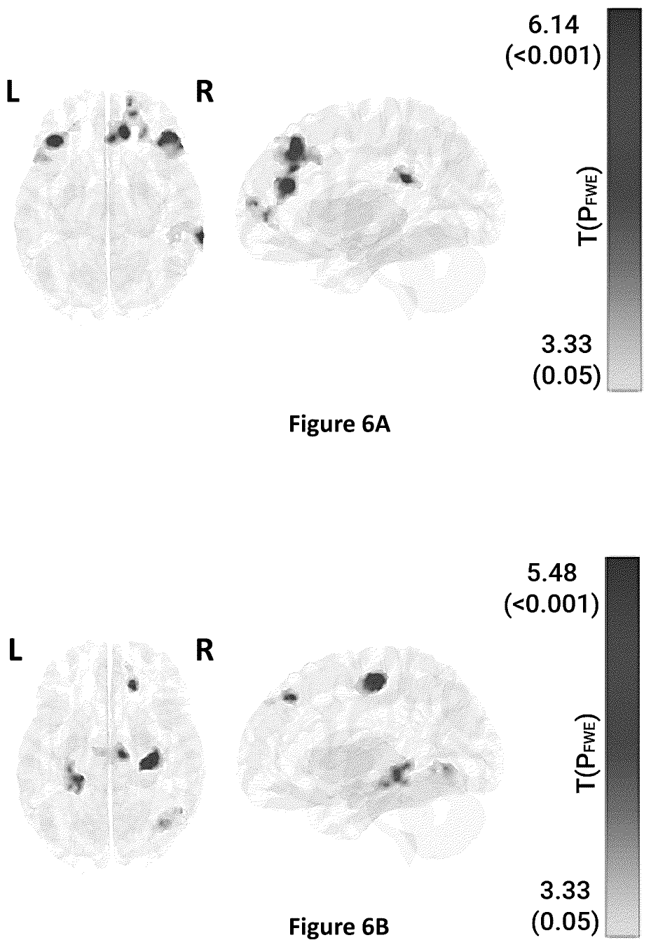

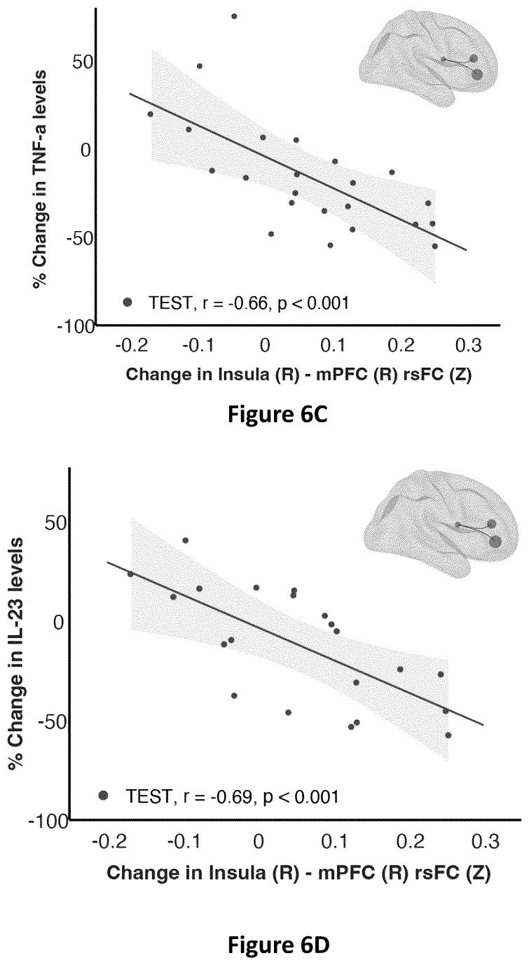

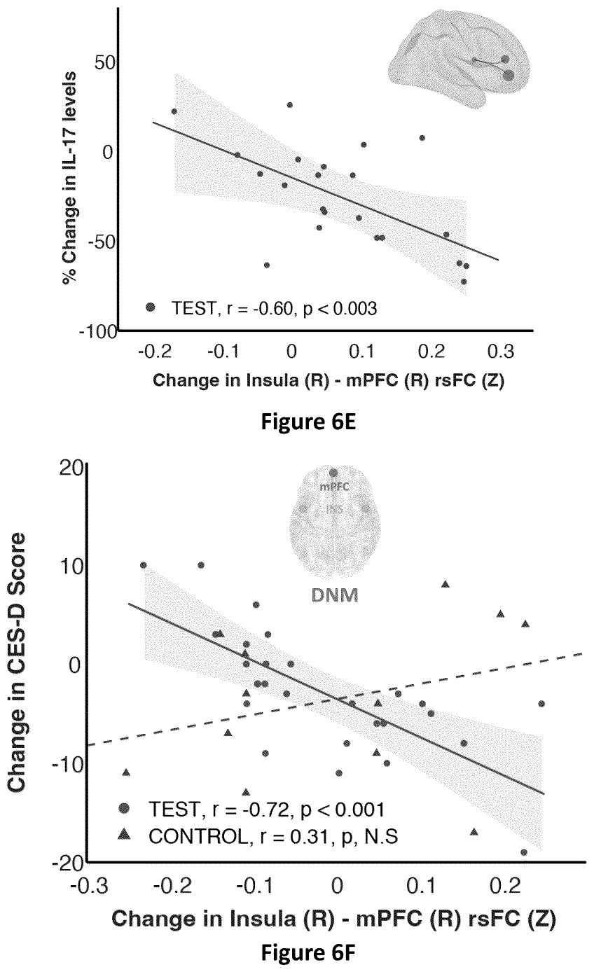

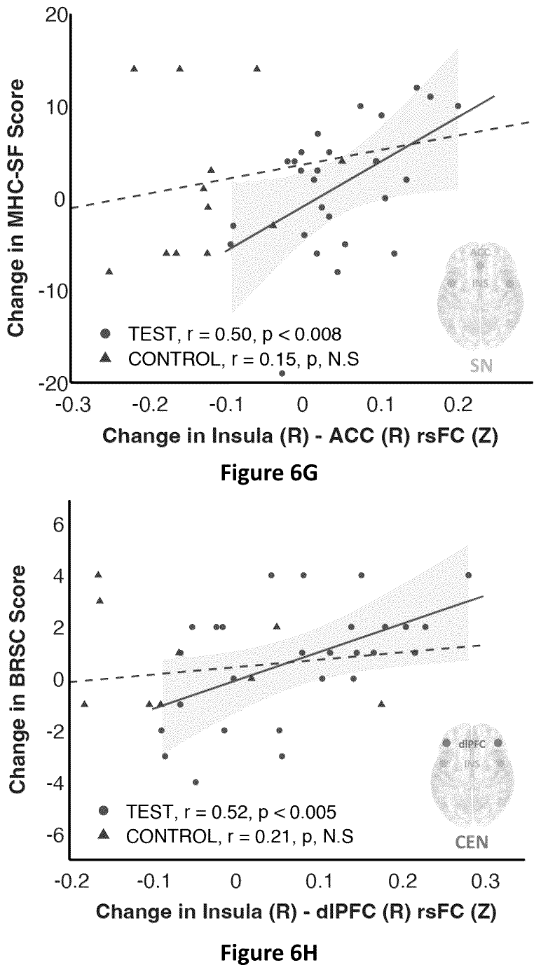

FIGS. 6A-6H show the association between brain plasticity, peripheral inflammation, and psychological state. In FIGS. 6A-6B, regarding brain connectivity: Group-by-time differences in rsFC between insula (R/L) and mPFC (voxel p<0.001, cluster p<0.05, FWE-corrected) suggest insula's key role in the brain-immune axis (n=27). In FIGS. 6C-6E regarding the inflammation link: Increased insula-mPFC rsFC was significantly associated with reduced IL-23, IL-17, and TNF-α levels (voxel p<0.001, FDR-corrected). In FIGS. 6F-6H regarding psychological improvement: rsFC changes in insula-mPFC, ACC, and dlPFC correlated with improved depression, mental health, and resilience scores (voxel p<0.001, FDR-corrected). Thus, the mobile intervention led to significant improvements in psychological state and immune function. Combined behavioural, neural, and immune measures revealed early mechanisms of change, highlighting the role of fronto-limbic circuits, especially the insula and amygdala.

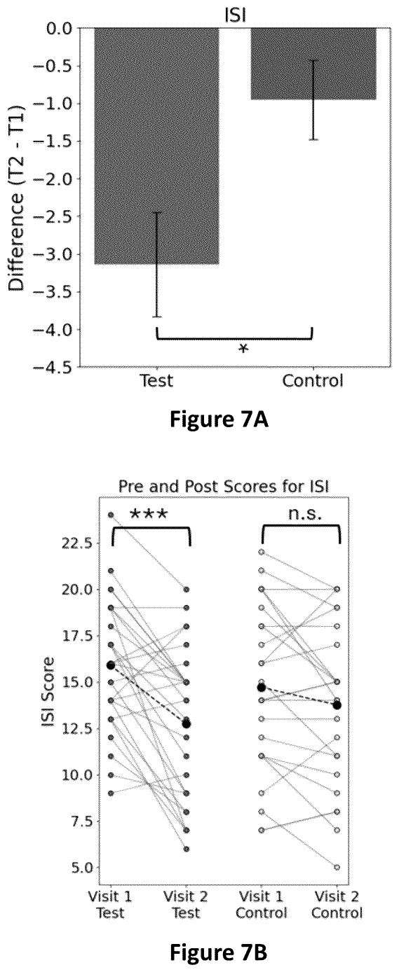

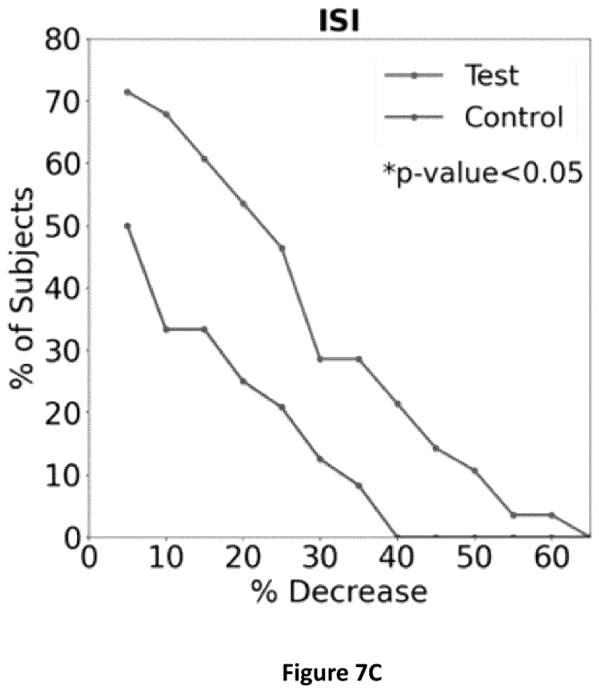

FIGS. 7A-7C show significant improvements in the ISI scores between subject with sleep difficulties. The ISI is a 7-item scale focusing on insomnia symptom severity and impact over the past 2-4 weeks. Items rate difficulty falling asleep, staying asleep, early morning awakening, satisfaction with sleep, interference with daytime functioning, noticeability of impairment, and distress about sleep problems. Thus the digital interventions of the invention improved insomnia symptoms.

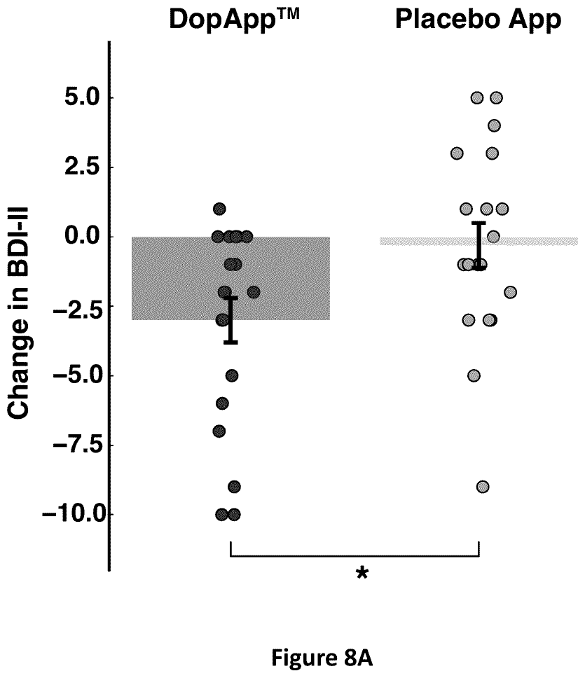

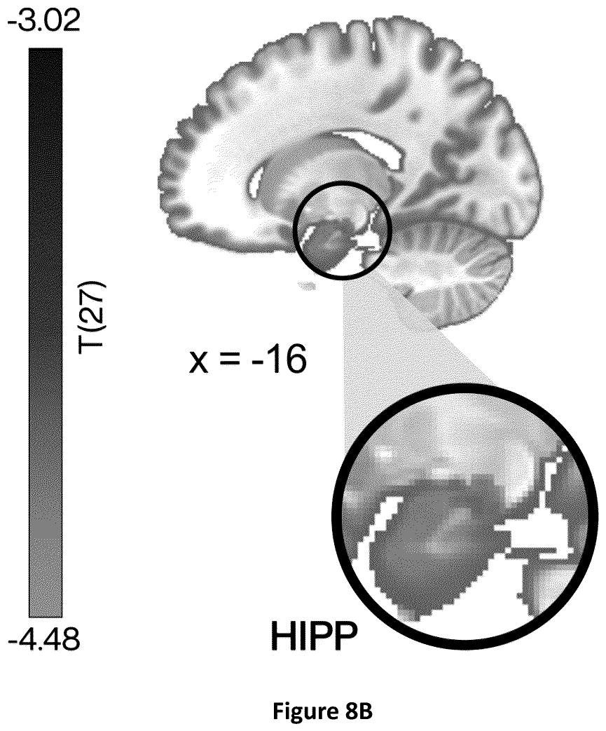

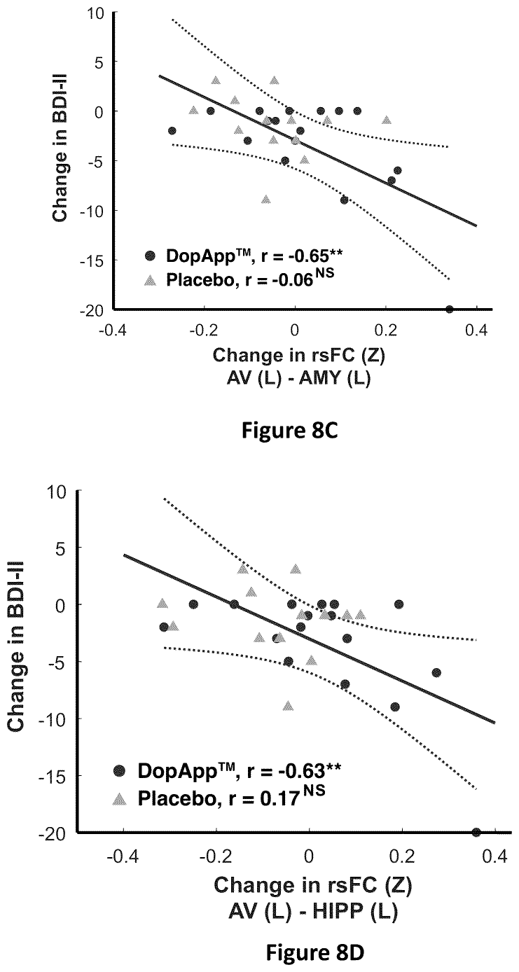

FIGS. 8A-8D shows results from a Parkinsons disease study which uses the digital interventions of the invention. There was a post intervention improvement in depressive symptoms, Beck's Depression Inventory II (BDI-II) scores. This improvement was correlated with brain connectivity changes within the limbic circuit.

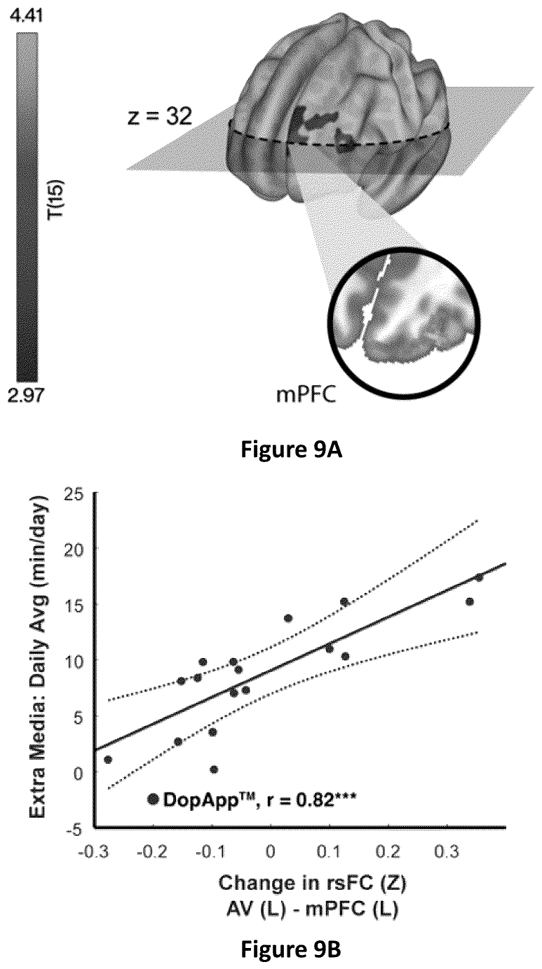

FIGS. 9A-9B show that, regarding app usage, there is an increased rsFC between the left AV and the medial prefrontal cortex was correlated with higher self-initiated daily doses of emotion regulation content usage. This neural pathway, which is part of the DMN, is implicated in self-referential processing and emotional regulation. Thus the software application (also referred to as the digital app, app, or DopApp) use improved depressive symptoms with correlation to brain connectivity changes. Higher self-engagement was correlated with connectivity changes in related brain networks. Engagement with specific app modules predicted corresponding clinical improvements and associated connectivity changes, indicating dose-dependent, and circuit-specific neuroplasticity.

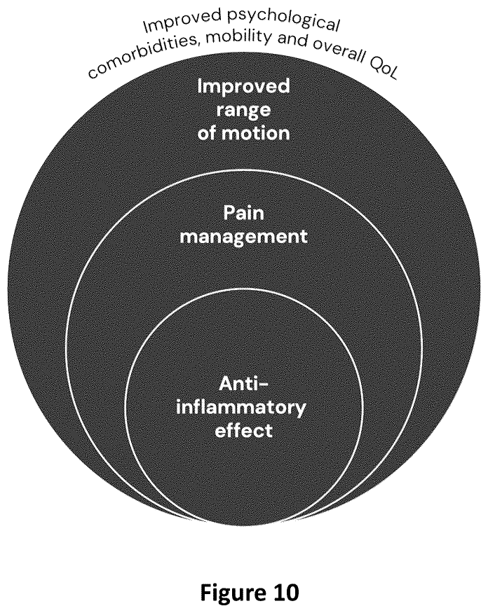

FIG. 10 shows a schematic representation of a hybrid drug effect for TGCT comprising improved psychological comorbidities, mobility and overall quality of life. The sphere diagram shows that the software application of the invention works, at least, by eliciting an anti-inflammatory effect. The reduced inflammation results in diminished pain, which in turn leads to a greater range of motion of the affected joint(s). However, unlike chemical drugs, the software application of the invention is multi-modal and contains specific interventions aimed directly at reducing pain (by increasing pain tolerance and blunting pain perception) and increasing range of motion (through physical therapy). In other words, the methods of the invention affect the secondary endpoints (e.g., pain, stiffness, range of motion, physical function, and quality of life indicators) both as a consequence of reduced inflammation and as a direct response to some of the interventions included in the software application. This leads to functional improvements and fewer side effects.

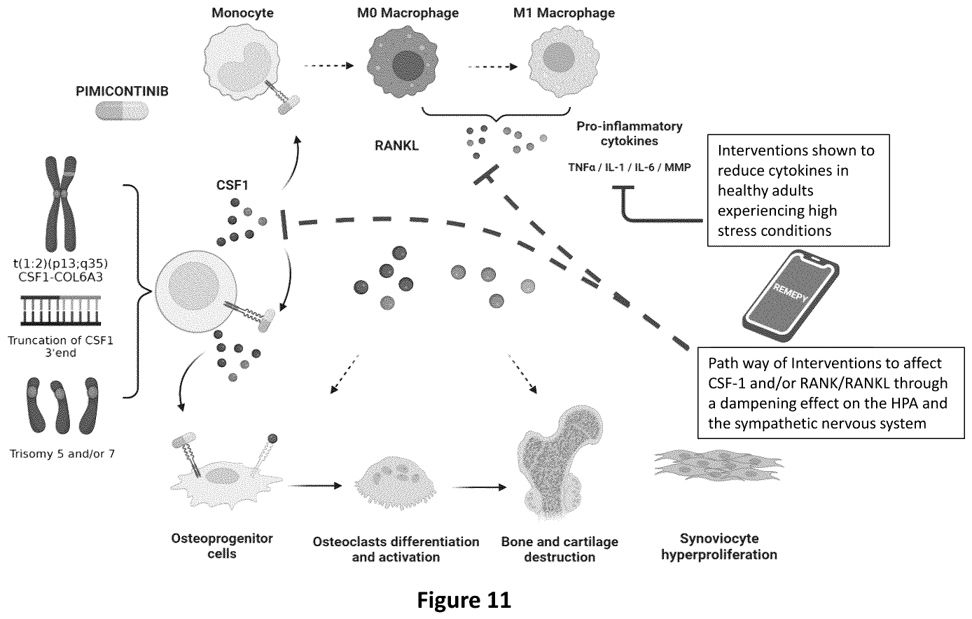

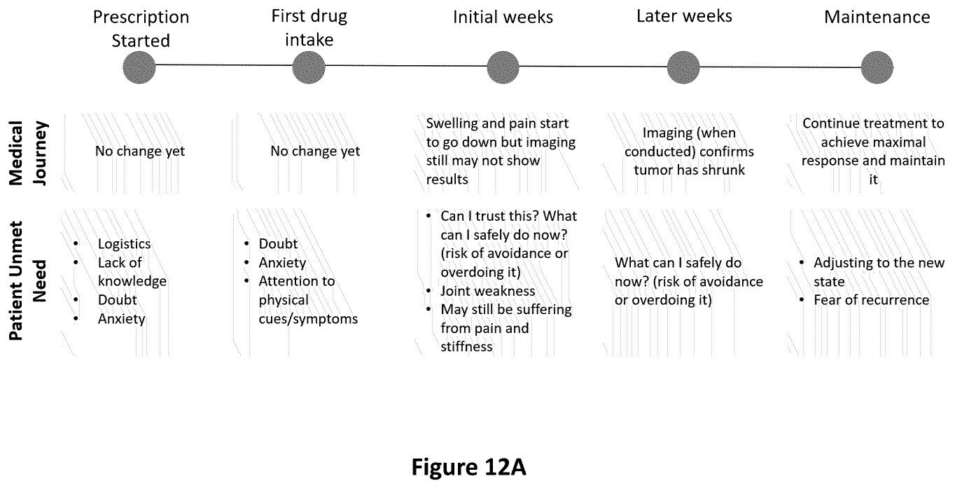

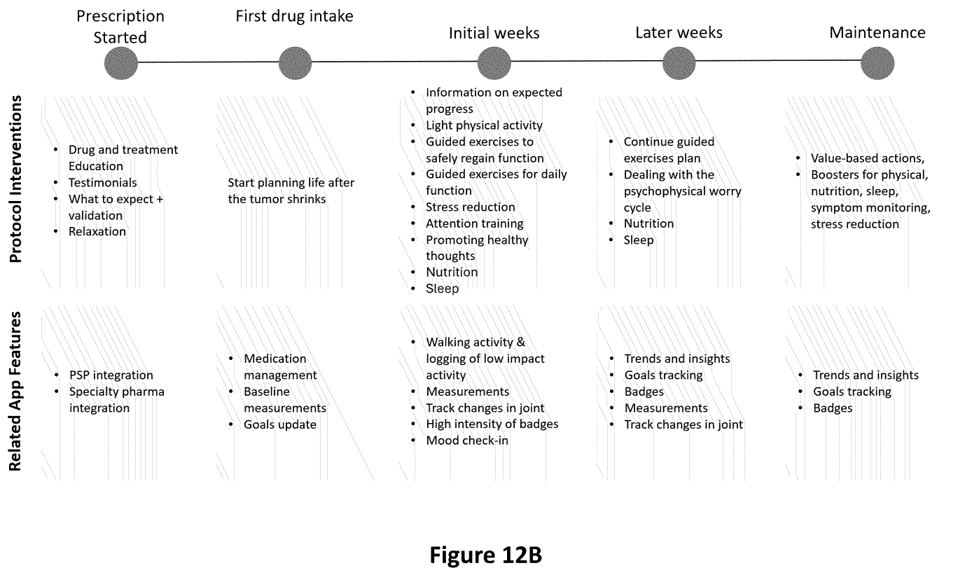

FIG. 11 illustrates the specific inflammatory components targeted by the software application of the invention, as well as those influenced by CSF1R blockers, in producing the overall anti-inflammatory effect. Because TGCT tumors consist largely of inflammatory cells recruited to the joint, reducing these inflammatory mediators is expected not only to alleviate inflammation but also to decrease tumor size, which serves as the primary endpoint.

### Example 2 Patient Insights and Highlights

Diagnosis & Personal Journey. Patients often faced misdiagnosis, with conditions like arthritis or sports injuries initially suspected, and imaging delayed or poorly explained. The diagnostic process stretched over months or years, causing anxiety and mistrust, while the eventual diagnosis brought both relief and fear. Many relied on self-advocacy and online research to find specialists and understand TGCT.

Treatment Decisions & Experiences. Surgery was usually the first treatment choice, but recurrence shifted attitudes toward systemic therapy. While side effects such as fatigue or liver issues were noted, systemic treatment often reduced swelling and pain, offering emotional reassurance and improved quality of life.

Physical Activity & Exercise. Movement was a central coping strategy, supporting both physical recovery and emotional resilience. Patients redefined exercise by celebrating small wins, though they struggled with fear, frustration, and limited TGCT-aware physiotherapy. They expressed a need for guided pacing tools, adjustable plans, and motivational reinforcement.

Pain & Daily Functioning. Pain was constant, unpredictable, and exhausting, often worse after surgery than expected. Sleep and pain were tightly linked, creating cycles of fatigue and anxiety. Patients carefully balanced movement and rest, relying on pacing routines that demanded mental effort but helped manage swelling and stability.

Mental Health & Identity. Emotional strain was widespread, with anxiety, loss of independence, and career disruptions common. Pain strongly influenced mood, leading to cognitive fog and diminished confidence. Patients found resilience through mindfulness, self-talk, and acceptance, and showed interest in mental health tools like journaling and meditation.

Life Hacks & Daily Adaptations. Patients developed creative strategies to manage pain and fatigue, such as compression sleeves, ice therapy, adaptive shoes, and step tracking. Peer-shared advice and simple environmental adjustments were highly valued for improving daily functioning.

Nutrition & Self-Care. Nutrition was seen as part of holistic recovery, though pain and immobility often disrupted healthy routines. Some patients used food for stability and empowerment, adopting anti-inflammatory diets or cooking as therapy. They expressed interest in app-based reminders for hydration, balanced meals, and energy tracking.

In one embodiment, the term “a” or “one” or “an” refers to at least one. In one embodiment the phrase “two or more” may be of any denomination, which will suit a particular purpose. In one embodiment, “about” or “approximately” may comprise a deviance from the indicated term of +1 %, or in some embodiments, −1 %, or in some embodiments, ±2.5 %, or in some embodiments, ±5 %, or in some embodiments, ±7.5 %, or in some embodiments, ±10 %, or in some embodiments, ±15 %, or in some embodiments, ±20 %, or in some embodiments, ±25 %.

Those skilled in the art to which this invention pertains will readily appreciate that numerous changes, variations, and modifications can be made without departing from the scope of the presently disclosed subject matter, mutatis mutandis.

1. A method for modulating one or more disease-related markers, symptoms, or disease progression in an individual with benign connective tissue tumor originating from, or causing, dysregulated immune response, the method comprising:
delivering a digitized therapeutic plan to the individual, wherein the digitized therapeutic plan comprises a plurality of therapeutic interventions selected from: at least one physical intervention and at least one psychological intervention;
converting the plurality of therapeutic interventions into a plurality of targeted interaction outputs for presentation to the individual; and
adaptively delivering the digitized therapeutic plan, tracking the individual's responses and associated performance metrics, and continuing until a predetermined condition is met.

2. The method of claim 1 wherein the benign connective tissue tumor is Tenosynovial Giant Cell Tumor (TGCT).

3. The method of claim 1 further comprising obtaining a plurality of attributes associated with the individual from a plurality of data acquisition sources to generate an initial profile for the individual, wherein the digitized therapeutic plan further comprises adaptation based on the initial profile.

4. The method of claim 1 wherein the digitized therapeutic plan comprises performing the at least one physical intervention concurrently with the at least one psychological intervention, or sequentially, or a combination thereof.

5. The method of claim 3 wherein the adaptation further comprises selecting and implementing therapeutic intervention parameters based on at least one attribute, of the plurality of attributes, of the individual selected from: age, hobbies, music preferences, spoken language, caregiver involvement preferences, disease characteristics, tumor location, tumor size, disease severity, the disease-related markers, the symptoms, pain levels, range of motion limitations, joint stiffness, educational background, professional background, and individual-defined goals.

6. The method of claim 5 wherein the digitized therapeutic plan is further dynamically adapted based on changes to at least one of the plurality of attributes of the individual during the course of the digitized therapeutic plan.

7. The method of claim 3 further comprising:
dynamically monitoring the individual's monitored parameters during delivery of the digitized therapeutic plan;
generating a dynamic user profile based on the initial profile and monitored parameters of the individual selected from: Electronic Medical Records (EMR) data, motor attributes, psychological attributes, range of motion measurements, pain levels, joint function, pharmacological attributes, tumor response parameters, disease progression, feedback of the individual, feedback of a medical practitioner, drug intake parameters, or a combination thereof; and
adapting the digitized therapeutic plan based on the dynamic user profile.

8. The method of claim 7 further comprising recursively adapting the digitized therapeutic plan based on updates to the dynamic user profile, the performance metrics, and the individual's responses.

9. The method of claim 8 wherein the recursive adapting of the digitized therapeutic plan comprises dynamically identifying and modifying the therapeutic intervention parameters to implement optimized therapeutic outcomes towards a predetermined therapeutic goal.

10. The method of claim 9 wherein the therapeutic intervention parameters comprise any of the following selected from: type, timing, dosage, intensity, sequence, duration, frequency, mode of delivery, level of guidance, feedback modality, language adaptation, complexity level, interface configuration, integration with external systems, or a combination thereof, of one or more of the therapeutic interventions.

11. The method of claim 8 wherein the recursive adapting is performed using a feedback control algorithm, Bayesian inference, or a combination thereof.

12. The method of claim 1 wherein the at least one physical intervention is selected from: physiotherapy, range-of-motion exercises, mobility training, limb agility exercises, functional movement training, balance exercises, proprioception exercises, postural stability training, strength training, stretching exercises, coordination training, low impact training, aerobics exercises, flexibility training, and walking, or a combination thereof.

13. The method of claim 1 wherein the at least one psychological intervention is selected from: guided imagery, psychoeducation, psychotherapy, cognitive behavioral therapy, stress and anxiety management training, mindfulness-based interventions, body scanning training, sleep hygiene, fatigue training, pain psychology, pacing training, acceptance and commitment therapy (ACT), dialectical behavior therapy (DBT), attention training techniques, psychodynamic therapy, solution-focused brief therapy (SFBT), narrative therapy, pain therapy, addiction therapy, gestalt therapy, behavioral activation therapy, adjustment therapy, grief therapy, motivational therapy, meaning-centered therapy, creative therapy, expressive therapy, logotherapy, telepsychiatry, and teletherapy, or a combination thereof.

14. The method of claim 1 wherein each of the plurality of therapeutic interventions has a duration ranging between 10 s to 60 mins.

15. The method of claim 1 wherein the predetermined condition is selected from: a predetermined number of iterations, a predetermined duration has elapsed, a predetermined number of therapeutic interventions have been completed, predetermined progress measurement, predetermined regression measurement, target threshold score achieved, transition to maintenance therapy, and no further improvement is measured, or a combination thereof.

16. The method of claim 1 wherein the targeted interaction outputs are selected from: visual, auditory, tactile, or a combination thereof.

17. The method of claim 1 wherein the plurality of therapeutic interventions further comprise: sensory inhibition, sensory substitution, sensory integration, or a combination thereof.

18. The method of claim 1 further comprising pairing a conditioned stimulus with at least one therapeutic intervention such that subsequent presentation of the conditioned stimulus elicits a therapeutic response associated with the intervention.

19. The method of claim 1 further comprising implementing a nutrition regimen in the individual.

20. The method of claim 19 wherein the nutrition regimen comprises any of the following selected from: portion sizing to maintain healthy body weight, meal timing, anti-inflammatory diet, joint-supportive nutrients, weight management support, reduction of pro-inflammatory food intake, hydration, Mediterranean diet, or a combination thereof.

21. The method of claim 1 wherein the disease-related markers comprise any of the following selected from: tumor size, tumor spread, objective response rate (ORR), inflammation, macrophage recruitment or activity, cytokine levels, angiogenesis, tumor cell proliferation, fibrosis, joint swelling, pain, stiffness, physical function, range of motion, quality of life, patient perception of disease, psychological endpoints, grip strength, and gait stability.

22. The method of claim 1 wherein the benign connective tissue tumor is selected from: TGCT, lipoma, angiolipoma, fibrolipoma, spindle cell lipoma, pleomorphic lipoma, hibernoma, fibroma, desmoid tumor, nodular fasciitis, palmar fibromatosis, plantar fibromatosis, elastofibroma dorsi, myofibroma, leiomyoma, angioleiomyoma, rhabdomyoma, schwannoma, neurofibroma, perineurioma, traumatic neuroma, hemangioma, lymphangioma, glomus tumor, myxoma, intramuscular myxoma, granular cell tumor, solitary fibrous tumor, Giant Cell Tumor of Bone (GCTB), histiocytic disorders, lipoma arborescens, and synovial chondromatosis.

23. The method of claim 1 further comprising administering at least one medicinal agent before, during, after, or a combination thereof, the implementation of at least one of the therapeutic interventions.

24. The method of claim 23 wherein the at least one medicinal agent comprises any of the following selected from: CSF1R-inhibitor, anti-inflammatory drug, immunomodulatory drug, cytokine-targeting biologic, nirogacestat, denosumab, everolimus, methotrexate, and corticosteroids or a combination thereof.

25. The method of claim 24 wherein the CSF1R-inhibitors comprises any of the following selected from: Pimicotinib, Pexidartinib, Vimseltinib,, BLZ945 (Sotuletinib), JNJ-40346527 (Edicotinib), IACS-9439, BPR1R024, ABT-869 (linifanib), Imatinib, AG013736 (axitinib), SU11248 (sunitinib), BAY 43-9006 (sorafenib), CHIR258 (tandutinib), Ki20227, Emactuzumab (RG7155), Lacnotuzumab (MCS110), Cabiralizumab (FPA008), PD-036032, ARRY-382 (PF-07265804), EI-1071, TE-952 (JTE-952), PLX5622, GW2580, IMC-CS4 (LY3022855), and AMG820 or a combination thereof.

26. The method of claim 23 further comprising the administration of an adjunct agent comprising any of the following selected from: nonsteroidal anti-inflammatory drugs (NSAIDS), immunomodulatory agent, cytokine-targeting biologic, corticosteroids, analgesics, anesthetic agents, or a combination thereof.

27. The method of claim 23 further comprising a conditioning stimulus delivered before, during, after, or a combination thereof, the administering of the at least one medicinal agent, at least one of the therapeutic interventions, or a combination thereof.

28. The method of claim 23 further comprising:
obtaining a plurality of attributes associated with the individual from a plurality of data acquisition sources to generate an initial profile for the individual, wherein the digitized therapeutic plan further comprises adaptation based on the initial profile;
wherein the adaptation further comprises selecting and implementing therapeutic intervention parameters based on at least one attribute of the individual selected from: age, hobbies, music preferences, spoken language, caregiver involvement preferences, disease characteristics, tumor location, tumor size, disease severity, disease-related markers, symptoms, pain levels, range of motion limitations, joint stiffness, educational background, professional background, medicinal agent intake parameters, and individual-defined goals.

29. A digital therapeutic system for modulating one or more disease-related markers, symptoms, or disease progression in an individual with benign connective tissue tumor originating from, or causing, dysregulated immune response, the system comprising:
a data-input module configured to onboard the individual and dynamically monitor and store data related to the individual throughout a digitized therapeutic plan; and
a digital therapy delivery module comprising a processor configured to deliver the digitized therapeutic plan comprising a plurality of therapeutic interventions selected from: at least one physical intervention and at least one psychological intervention;
wherein the digital therapy delivery module is further configured to convert the digitized therapeutic plan into a plurality of targeted interaction outputs for presentation to the individual and track the individual's responses.

30. The system of claim 29 wherein the digital therapy delivery module comprises software executable on a personal electronic device.

31. The system of claim 29 wherein the data-input module is configured to receive data from a plurality of data acquisition sources in real-time, wherein the data comprises one or more of: Electronic Medical Records (EMR) data, motion sensor data, position sensor data, environmental sensors data, audio sensors data, location sensors data, audio recording data, speech recognition data, gaze-tracking data, touch interaction data, physiological sensor data, optical sensor, imaging sensors, self-report data, or a combination thereof.

32. The system of claim 29 wherein the digital therapy delivery module comprises any of the following selected from: a personal electronic device, a graphical user interface, audio interface, haptic interface, or a combination thereof.

33. The system of claim 29 wherein the processor is further configured to:
dynamically monitor any of the following factors of the individual selected from: Electronic Medical Records (EMR) data, motor attributes, psychological attributes, cognitive attributes, pharmacological attributes, and drug intake parameters, or a combination thereof, throughout the implementation of the digitized therapeutic plan;
generate a dynamic user profile, and recursively adapt the digitized therapeutic plan based on the dynamic user profile, performance metrics, and the individual's responses;
or a combination thereof.

34. The system of claim 29 further comprising at least one additional device operatively coupled to the digital therapy delivery module, selected from: health monitoring systems, medical devices, haptic devices, external speakers, headphones, virtual reality headsets, augmented reality glasses or devices, biofeedback sensors, wearable activity trackers, smartphones, tablets, smartwatches, personal computational devices, smart speakers, voice assistants, motion tracking sensors, virtual assistant systems, internet hubs, gait sensors, eye-tracking devices, voice recording devices, and wearable devices, or combinations thereof.
